(19) United States
(12) Patent Application Publication
(10) Pub. No.: US 2026/0275578 A1
(43) Pub. Date: Sep. 17, 2026

(54) METHOD OF PRODUCTION OF FIBERS AND A DEVICE FOR CARRYING OUT THE METHOD

(71) Applicant: CONTIPRO A.S., Dolni Dobrouc (CZ)

(72) Inventors: Tomas Medek, Usti nad Orlici (CZ); Marek Pokorny, Cenkovice (CZ); Jan KLEMES, Moravska Trebova (CZ); Vladimir VELEBNY, Zamberk (CZ)

(73) Assignee: CONTIPRO A.S., Dolni Dobrouc (CZ)

(21) Appl. No.: 19/168,680

(22) PCT Filed: Mar. 27, 2024
(86) PCT No.: PCT/CZ2024/050017

§ 371 Date: Sep. 24, 2025

(30) Foreign Application Priority Data
Mar. 28, 2023 (CZ) PV2023-121

(51) Int. Cl. D01D 5/098; A61L 27/34; A61L 27/56; D01D 5/084; D01D 5/10

(52) U.S. Cl. CPC D01D 5/0985; A61L 27/34; A61L 27/56; D01D 5/084; D01D 5/10; D10B 2201/20; D10B 2321/021; D10B 2509/00

(57) ABSTRACT

A method is disclosed for the production of microfibres or nanofibres based on hyaluronic acid and/or a water-soluble metal or non-metal salt thereof or a mixture of salts and/or a derivative of hyaluronic acid by the method of dry spinning and/or solution blow spinning. Two-dimensional or three-dimensional fibrous materials may be formed from said microfibres and nanofibers, for example in the form of a layer or cotton wool. A device for carrying out the method is also disclosed. The device contains an extrusion piece containing a pass-through channel having an inlet opening for feeding the spinning solution and a dispensing opening for dispensing the spinning solution. The device further contains an air nozzle, the air outlet opening of which is arranged to direct the exiting air into the area surrounding the dispensing opening of the extrusion piece parallel to the axis of the dispensing opening of the extrusion piece.

### CROSS-REFERENCE TO RELATED APPLICATIONS

This application is the National Stage of International Application No. PCT/CZ2024/050017, filed on 27 Mar. 2024, which claims priority to and all advantages of CZ Application No. PV 2023-121, filed on 28 Mar. 2023.

### TECHNICAL FIELD

The present disclosure relates to a method and device for production of microfibres or nanofibres based on hyaluronic acid and/or its water-soluble metal or non-metal salt or a mixture of salts and/or its derivative by the method of dry spinning and/or solution blow spinning, and two-dimensional or three-dimensional fibrous materials made of these microfibres or nanofibres. Furthermore, the present disclosure relates to a device for performing this method.

### BACKGROUND

Hyaluronic acid (HA or hyaluronan) is a linear polysaccharide formed by repeating disaccharide units composed of D-glucuronic acid and N-acetylglucosamine according to the formula I

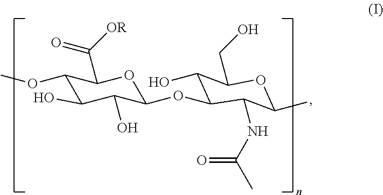

where R is H⁺ or a metal cation.

Hyaluronan is found in the intercellular spaces of most human tissues, where it influences a number of processes including the maintenance of homeostasis, regeneration and wound healing. Hyaluronan products have a variety of forms, such as injectable solutions and gels, foils or textiles. These products are used in medicine and cosmetics, for example, as medical devices for wound healing, treatment of osteoarthritis, prevention of postoperative adhesions or reduction of wrinkles.

In order to modify the properties of the native hyaluronan, a number of hyaluronan derivatives have been prepared in the past.

Hyaluronan chloramide is a derivative of hyaluronic acid in which most of the hydrogens of the amide group —NH—CO— are substituted by a chlorine atom to —NCI—CO—. The production thereof and the properties thereof, which include antimicrobial, antifungal and antiviral activity, are described in the document CZ 308010.

Crosslinkable hyaluronan derivatives are derivatives containing groups enabling the connection of polymer chains by covalent bonds. These include 3-(2-furanyl) acryloyl ester of hyaluronan and tyramine hyaluronan.

Hyaluronan 3-(2-furanyl) acryloyl ester can be crosslinked by UV radiation in the solid phase. The synthesis and electrospinning thereof are described in the document CZ 304977 B6.

Tyramine hyaluronan is the name for various conjugates of hyaluronan with tyramine, which can be used, for example, to prepare crosslinked hydrogels. The synthesis of tyramine hyaluronan according to Formula II is described in the document CZ 303879 B6. Crosslinking of tyramine hyaluronan using riboflavin and UV radiation is described in the publication Donnelly, P. E., Chen, T., Finch, A., Brial, C., Maher, S. A., & Torzilli, P. A. (2017). Photocrosslinked tyramine-substituted hyaluronate hydrogels with tunable mechanical properties improve immediate tissue-hydrogel interfacial strength in articular cartilage. Journal of Biomaterials Science, Polymer Edition, 28(6), 582-600.

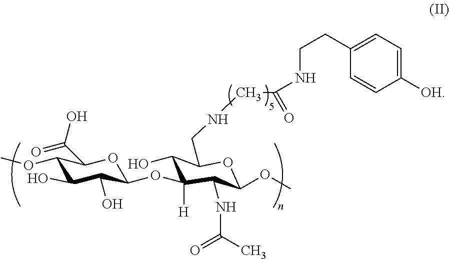

Non-polar derivatives of hyaluronan contain hydrophobic substituents. They can be classified to esters and acylated derivatives. The esters are hyaluronan benzyl ester and hyaluronan ethyl ester, their production is described in document U.S. Pat. No. 5,622,707.

The acylated derivatives are hyaluronan derivatives in which primarily the primary alcohol of N-acetyl-glucosamine and to a lesser extent the secondary alcohols of glucuronic acid are acylated with fatty acids. The acyl group can be, for example, caproyl (hexanoyl), capryloyl (octanoyl), caprinoyl (decanoyl), lauroyl (dodecanoyl), myristoyl (tetradecanoyl), palmitoyl (hexadecanoyl), stearoyl (octadecanoyl) and oleoyl (octadec-9-enoyl). Examples of the production of acylated derivatives are given in document WO 2014082611 A1.

In the field of spinning of hyaluronan and derivatives thereof, two types of technologies clearly prevail: electrospinning and wet spinning.

In electrospinning, the polymer solution is drawn into a fibre shape by the action of electrical forces. The production of fibres from an aqueous solution of hyaluronan by electrospinning is very difficult, therefore fibres are usually prepared from a mixture of hyaluronan with other polymers, for example polyethylene glycol or gelatin. Alternatively, pure hyaluronan can be spun by classical electrospinning when dissolved in a mixture of water and dimethylformamide or by the electroblowing method when dissolved in an aqueous solution of HCl at pH=1.5, which method uses, in addition to the electric field, an air stream that draws and dries the fibre (Lee, K. Y., Jeong, L., Kang, Y. O., Lee, S. J., & Park, W. H. (2009). Electrospinning of polysaccharides for regenerative medicine. Advanced Drug Delivery Reviews, 61(12), 1020-1032).

Hyaluronan fibres prepared by electrospinning are mostly deposited on a collector in the form of a thin non-woven fabric (two-dimensional structure). Document WO 2020/124072 A1 discloses a method of expanding two-dimensional nanofibrous layers into a three-dimensional structure by exposure to gas bubbles. Document CZ 2013-913 A3 describes the spinning conditions under which a bulky layer of nanofibres is directly formed.

One of the drawbacks of the hyaluronan electrospinning technology is its limitation to the production of fibres of small diameters, in most cases less than 1 micrometer. Such fibres are exemplified by low stiffness, low strength and a very fast dissolution in an aqueous environment. Furthermore, the solvents used for spinning pure hyaluronan are either unsuitable for use in healthcare due to their toxicity (e.g., dimethylformamide), or cause hyaluronan degradation (e.g., acid hydrolysis in an aqueous HCl solution). In case of using a mixture of hyaluronan with another polymer, the overall properties of the material change.

The principle of wet spinning of a polymer is the extrusion of a polymer solution into a coagulation bath, in which the coagulation (precipitation) of the polymer into a fibre form occurs and the original solvent diffuses bidirectionally from the fibre into the coagulation bath and from the coagulation bath into the fibre. For example, the document U.S. Pat. No. 8,753,671 B2 discloses the production of an endless fibre (filament) from hyaluronan by wet spinning. The document WO 94/17837 discloses the production of non-woven fabrics from staple fibres obtained by cutting the hyaluronan ester filament prepared by wet spinning, the cohesion of these fibres is additionally improved by chemical bonding. The document CZ 304651 B6 describes the direct production of staple fibres by hyaluronan spinning in a non-stationary coagulation bath and their subsequent processing into a non-woven fabric with steps including at least filtration and drying of the fibres.

The main disadvantage of the wet spinning technology is the two-way mass transfer during the fibre precipitation, which makes the fibre formation process slower than in other types of spinning. After removing the fibres from the coagulation bath, it is necessary to dry them from the residues of the coagulation bath, often the residues of the low-volatile components of the coagulation bath must be washed out of the fibres first. In case of hyaluronan spinning, acid coagulation baths are most often used, in which acid hydrolysis of hyaluronan chains occurs and hyaluronan salts are converted to an acidic form. Non-woven fabrics from hyaluronan are not prepared directly by wet spinning, but the obtained fibres must be processed into their form in further steps by one of the known procedures, typically wet or dry way of the production of non-woven fabrics.

Due to the aforementioned disadvantages of wet spinning, dry spinning or even better melt spinning is preferred when spinning polymers. The principle of melt spinning is the extrusion of the polymer melt into a cooling gas, where the melt stream solidifies into fibres. Therefore, neither a coagulation bath nor a solvent is present here, and the solid fibre is formed from the liquid state very quickly, since the process is not slowed down by mass transfer. However, hyaluronic acid cannot be melted, because due to the presence of strong intermolecular bonds between its chains, it degrades, when heated, before it starts to melt. The same applies to most of its derivatives.

Only WO 2005/028632 A2 discloses the production of hyaluronan esters in which aliphatic acyls disrupt the intermolecular bonds to such an extent that the hyaluronan derivative becomes meltable. The document WO 2017/039335 A1 discloses the production of hyaluronan fibres using a melt spinning device, where the fibre is formed from hyaluronan comprising 5 to 20% of water at 150 to 200° C. and is subsequently cured in mixtures of water and ethanol, so it is not a true melt spinning.

The principle of dry spinning is the extrusion of a polymer solution into a drying gas, in which the solution is dried into fibres. There is a one-way transfer of mass, solvent from the solution to the drying gas, and therefore dry spinning is between the wet spinning and the melt spinning in terms of difficulty and speed of production. Dry spinning of hyaluronan is not known yet. Snetkov in his comprehensive article (Snetkov, P., Morozkina, S., Uspenskaya, M., & Olekhnovich, R. (2019). Hyaluronan-Based Nanofibers: Fabrication, Characterization and Application. Polymers, 11(12), 2036) reported that dry spinning of hyaluronan is difficult to be carried out due to the solubility of hyaluronan in water and could probably be carried out for hydrophobic hyaluronan derivatives that are soluble in easily evaporable organic solvents.

The document WO 89/10941 A1 refers to a production and processing of crosslinked acidic polysaccharide esters including hyaluronan and as one of the options for spinning thereof mentions the dissolution of the crosslinked polysaccharide in an organic solvent and—if the solvent used does not have a very high boiling point-removal of the solvent by a dry spinning process. In Examples, however, only the wet spinning of cross-linked carboxymethyl cellulose is described.

The document JP 2007-262595 A, which is the closest to dry spinning of hyaluronan, discloses production of an ultrathin filament from crosslinked hyaluronan, which is prepared by extruding a crosslinked gel and its subsequent mechanical stretching and pulling in an oven.

The document WO 2018/235745 A1 describes the production of non-woven fabrics by extruding a heated polymer solution that is captured in an air stream coming from a nozzle that is not in contact with the extrusion nozzle. The document discourages the arrangement where the air flows around the extrusion nozzle. The air carries the solution to the collector, where it is deposited in the form of partially dried fibres that need to be dried by lyophilization, which in Examples takes 72 hours, which significantly complicates and prolongs the process. In addition, the document mainly focuses on the spinning of gelatin, for which there are all examples, hyaluronan is only mentioned, without any parameters.

Centrifugal solution spinning is based on a principle similar to dry spinning. The principle is the extrusion of the solution by centrifugal force through the openings in the walls of a rotating container, the stream of the extruded solution is then drawn into the form of a fibre by the action of centrifugal force and frictional force caused by air resistance, and the resulting fibres are collected on a circular collector, in the centre of which the rotating container is located. The document US 2013/0312638 A1 discloses a device for centrifugal spinning of polymers, but does not give any examples or specific parameters for hyaluronan. The document CN 110424059 A discloses centrifugal spinning of blends of biopolymers with auxiliary polymers (e.g. polyethylene oxide, polyvinyl alcohol and polyvinylpyrrolidone), but does not provide any examples or specific parameters for hyaluronan. The document WO 2014/127099 A2 then states that centrifugal spinning in a classical arrangement is not suitable for solutions with slowly evaporating solvents (such as water) and recommends for them a technology called “immersed rotary jet spinning”, which differs from the classical centrifugal spinning by bath of the liquid into which the fibres are deposited. So, it is a kind of combination of centrifugal and wet spinning. Even this document only mentions hyaluronan and does not give any examples or parameters. Chantre in his publication describes the use of “immersed rotary jet spinning” technology, directly for spinning hyaluronan (Chantre, C. O., Gonzalez, G. M., Ahn, S., Cera, L., Campbell, P. H., Hoerstrup, S. P., & Parker, K. K. (2019). Porous Biomimetic Hyaluronic Acid and Extracellular Matrix Protein Nanofiber Scaffolds for Accelerated Cutaneous Tissue Repair. ACS Applied Materials & Interfaces, 11(49), 45498-45510).

The document CN 110424059 A recommends the addition of another polymer (e.g., polyethylene glycol or polyvinyl alcohol) for spinning biopolymers by centrifugal spinning, similarly to the case of electrospinning.

A relatively new polymer solution spinning technology first described by Medeiros in 2009 (Medeiros, E. S., Glenn, G. M., Klamczynski, A. P., Orts, W. J., & Mattoso, L. H. C. (2009). Solution blow spinning: A new method to produce micro- and nanofibers from polymer solutions. Journal of Applied Polymer Science, 113(4), 2322-2330) is solution blowing, also called solution blow spinning. The principle thereof is to dose a polymer solution into a stream of high-speed unheated gas through a needle located coaxially in an air nozzle. The publication describes the processing of synthetic polymers dissolved in organic solvents into fibres with diameters from hundreds of nanometers to units of micrometers. In document U.S. Pat. No. 8,641,960 B1, the same author also mentions spinning of other polymers, e.g. polyethylene oxide, polyvinylpyrrolidone and polyvinyl alcohol, by this technology.

Document IN 201741017782 A discloses a simple device for solution blow spinning and lists hyaluronan derivatives in the list of spinnable materials, but does not specify any specific parameters for them or which derivatives they are.

### BRIEF SUMMARY

Methods of producing fibres based on a polymer selected from hyaluronic acid, a water-soluble metal compound thereof, a derivative thereof, or combinations thereof, by dry spinning and/or solution blow spinning, are provided herein. Devices for carrying out the method are also provided herein. In an embodiment, a method comprises the steps of providing a spinning solution and extruding the spinning solution by an extrusion piece having at least one opening having a diameter of from about 80 to about 410 μm with a rate of from about 0.01 to about 0.7 mL/min into a drying air stream to form fibers that are carried to a collector. The spinning solution comprises from about 0.08 to about 3% by weight of hyaluronic acid, a water-soluble metal or non-metal salt thereof or a water-soluble mixture of metal and/or non-metal salts of hyaluronic acid and/or hyaluronic acid derivative, having a high molecular weight in the range of from about 1.1 MDa to about 2.5 MDa. The spinning solution further comprises from about 0.1 to about 22% by weight of hyaluronic acid, a water-soluble metal or non-metal salt thereof or a water-soluble mixture of metal and/or non-metal salts of hyaluronic acid and/or hyaluronic acid derivative, having a low molecular weight in the range of from about 5 kDa to about 750 kDa. The water-soluble metal or non-metal salt of hyaluronic acid is selected from the group comprising Na⁺, K⁺, Li⁺, Ca²⁺, Mg²⁺, Ag⁺, Au⁺, Zn²⁺, Cu²⁺, and NH⁴⁺ salt of hyaluronic acid, and the hyaluronic acid derivative is selected from the group comprising hyaluronan chloramide, hyaluronan 3-(2-furanyl) acryloyl ester, hyaluronan tyramine, hyaluronan benzyl ester, hyaluronan ethyl ester, and acylated hyaluronan derivatives selected from the group comprising capronoyl, capryloyl, caprinoyl, myristoyl, palmitoyl, stearoyl, and oleoyl hyaluronan. The spinning solution further comprises from about 0 to about 50% by weight of organic solvent and from about 48 to about 95% by weight of water.

### BRIEF DESCRIPTION OF THE DRAWINGS

FIG. 1 illustrates an embodiment of a device with a heat gun (not shown) as an air source.

FIG. 2 illustrates an embodiment of a device with a compressor as an air source and with an extrusion conical needle coaxially located in the centre of the air nozzle.

FIG. 3A illustrates an embodiment of a device with the combination of non-heated and heated air.

FIG. 3B illustrates a detailed section through the embodiment of the device with the combination of non-heated and heated air.

FIG. 3C illustrates a detailed view of a central part of the air nozzle of the embodiment of the device with the combination of non-heated and heated air.

FIG. 4 illustrates an embodiment of a device based on the principle of solution blow spinning technology.

FIG. 5 illustrates a rotary collector formed by bars.

FIG. 6 illustrates a stationary collector in the shape of an inverted cone, formed by bars.

FIG. 7 illustrates fibres comprising sodium hyaluronan prepared according to Example 1.

FIG. 8 illustrates fibres comprising sodium hyaluronan prepared according to Example 2.

FIG. 9 illustrates fibres comprising sodium hyaluronan prepared according to Example 4.

FIG. 10 illustrates fibres comprising sodium hyaluronan prepared according to Example 8.

FIG. 11 illustrates fibres comprising sodium hyaluronan prepared according to Example 9.

FIG. 12 illustrates fibres comprising a mixture of sodium hyaluronan and polyethylene oxide prepared according to Example 11.

FIG. 13 illustrates fibres comprising sodium hyaluronan prepared according to Example 13.

FIG. 14A illustrates cotton wool comprising a mixture of sodium hyaluronan and 3-(2-furanyl) acryloyl ester of hyaluronan prepared according to Example 15.

FIG. 14B illustrates a structure of crosslinked fibres comprising a mixture of 3-(2-furanyl) acryloyl ester of hyaluronan prepared according to Example 15 after 2 weeks in demineralized water.

FIG. 15 illustrates fibres comprising a mixture of sodium hyaluronan and hyaluronan chloramide prepared according to Example 16.

FIG. 16 illustrates fibres comprising a mixture of sodium hyaluronan and oxycellulose prepared according to Example 20.

FIG. 17 illustrates cotton wool comprising sodium hyaluronan, prepared according to Example 22.

FIG. 18A illustrates a non-woven textile comprising sodium hyaluronan on a polyester knitted fabric backing prepared according to Example 28.

FIG. 18B illustrates a detailed view of a non-woven textile comprising sodium hyaluronan on a polyester knitted fabric backing prepared according to Example 28.

FIG. 19A illustrates a pacemaker covered with a non-woven fabric comprising a mixture of sodium hyaluronan, Ponceau 4R, and Patent Blue VF prepared according to Example 29.

FIG. 19B illustrates fibres comprising a mixture of sodium hyaluronan, Ponceau 4R, and Patent Blue VF prepared according to Example 29.

FIG. 20A illustrates a pacemaker covered with a non-woven fabric comprising a mixture of sodium hyaluronan, Ponceau 4R, and Patent Blue VF prepared according to Example 30.

FIG. 20B illustrates fibres comprising a mixture of sodium hyaluronan, Ponceau 4R, and Patent Blue VF prepared according to Example 30.

FIG. 21 illustrates fibres comprising sodium hyaluronan prepared according to Example 31.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

It is to be appreciated that all values as provided herein, save for the actual examples, are approximate values with endpoints or particular values intended to be read as “about” or “approximately” the value as recited. Further, unless otherwise indicated, all amounts recited herein for components of the spinning solution are based on total weight of the spinning solution.

The drawbacks and limitations of the technical solutions mentioned in the foregoing background are solved by the present embodiments of the method of the production of fibres, where fibres are based on hyaluronic acid and/or water-soluble metal or non-metal salt thereof or on the basis of water-soluble mixture of metal and/or non-metal salts of hyaluronic acid and/or on the basis of hyaluronic acid derivative, where the essence of a method based on the principle of dry spinning and/or solution blow spinning is to prepare a spinning solution comprising hyaluronic acid and/or a metal or non-metal salt thereof or a mixture of salts and/or a hyaluronic acid derivative, which is then spun by extrusion into a drying air stream and the fibres are deposited on a collector in the form of a non-woven fabric. The present method is advantageous and differs significantly from the state of the art in that it allows simultaneously:
1. to spin hyaluronan without the addition of other polymers and using solvents acceptable in healthcare;
2. to spin hyaluronan without the use of a coagulation bath;
3. to direct the fibres to the collector by a stream of air without applying an electrical voltage;
4. to deposit the fibres on a collector directly in the form of both a flat and a bulky non-woven fabric of hyaluronan, which can be removed from the collector as a finished product immediately once the spinning is completed;
5. to dry the fibres on the collector in the order of units of up to tens of seconds, preferably of up to 30 seconds.

The present method differs from the experts' opinions in that it successfully uses the dry spinning principle to spin hyaluronan and its water-soluble derivatives.

The present disclosure therefore refers to the method of production of fibres based on hyaluronic acid and/or a water-soluble metal or non-metal salt thereof or on the basis of a water-soluble mixture of metal and/or non-metal salts of hyaluronic acid and/or on the basis of a hyaluronic acid derivative by the method of dry spinning and/or solution blow spinning, where a spinning solution of hyaluronic acid and/or a water-soluble metal or non-metal salt thereof or a water-soluble mixture of metal and/or non-metal salts of hyaluronic acid and/or hyaluronic acid derivative is prepared, containing 0.08 to 3% by weight of hyaluronic acid and/or its water-soluble metal or non-metal salt or a water-soluble mixture of metal and/or non-metal salts of hyaluronic acid and/or hyaluronic acid derivative, having a high molecular weight, 0.1 to 22% by weight of hyaluronic acid and/or its water-soluble metal or non-metal salt or a water-soluble mixture of metal and/or non-metal salts of hyaluronic acid and/or hyaluronic acid derivative, having a low molecular weight, 0 to 50% by weight of organic solvent, and 48 to 95% by weight of water, and after the complete dissolution of the polymer, the spinning solution is extruded through at least one opening for dispensing the solution, the opening having the diameter from 80 to 410 μm in the rate from 0.01 to 0.7 mL/min into the drying air stream, thereby obtaining fibres that are carried onto the collector. The water-soluble metal or non-metal salt refers for example to a compound of alkali metal, for example Na⁺, K⁺, Li⁺, or alkaline earth metals, such as Ca²⁺, Mg²⁺, or metals, such as Ag⁺, Au⁺, Zn²⁺, Cu²⁺, or non-metal, for example NH⁴⁺. The water-soluble mixture of metal and/or non-metal salts of hyaluronic acid can include in addition to monovalent metals or non-metals, bi- or tri-valent metal or non-metal salt. The hyaluronic acid derivative is preferably selected from the group comprising hyaluronan chloramide, hyaluronan 3-(2-furanyl) acryloyl ester, tyramine hyaluronan, hyaluronan benzyl ester, hyaluronan ethyl ester and acylated derivatives of hyaluronan selected from the group comprising capronoyl, capryloyl, caprinoyl, myristoyl, palmitoyl, stearoyl, oleoyl hyaluronan.

The weight average molecular weight of hyaluronic acid and/or the water-soluble metal or non-metal salts thereof and/or the derivative thereof, having a high molecular weight is in the range from 1.1 MDa to 2.5 MDa preferably in the range from 1.8 MDa to 2.5 MDa.

The weight average molecular weight of hyaluronic acid and/or the water-soluble metal or non-metal salts thereof and/or the derivative thereof, having a low molecular weight is in the range from 5 kDa to 750 kDa preferably in the range from 5 kDa to 100 kDa.

In a preferred embodiment the organic solvent is selected from the group comprising methanol, tetrahydrofuran, methyl acetate, methyl ethylketone, 1,2-dimethoxyethane, acetonitrile, isopropylalcohol, 1-propanol, ethanol and acetone, preferably isopropylalcohol in the amount from 25 to 40% by weight.

The spinning solution is preferably prepared by first dispersing hyaluronic acid and/or a water-soluble metal or non-metal salt thereof or a water-soluble mixture of metal and/or non-metal salts of hyaluronic acid and/or a hyaluronic acid derivative in an organic solvent, water is added to the resulting dispersion with thorough mixing, or hyaluronic acid and/or a water-soluble metal or non-metal salt thereof, or a water-soluble mixture of metal and/or non-metal salts of hyaluronic acid and/or a hyaluronic acid derivative is directly dissolved in water. Then the solution is stirred for 1 to 24 hours at temperature 20 to 30° C. until the polymer is completely dissolved. The prepared spinning solution is for example filled into a cartridge, which is sealed and connected to compressed air with an overpressure of +5 to +7 bar for 1 to 8 hours in order to dissolve the gas bubbles.

In another preferred embodiment, cooler air flows around the outlet opening for dispensing the solution, which air carries the spinning solution into a stream of warmer drying air. The drying air temperature is preferably 15 to 250° C., more preferably 15 to 160° C., and the absolute humidity of the drying air is preferably 0 to 14 g/m³, more preferably 0 to 2 g/m³, and the drying air flow rate is preferably 1.6 to 315 m/s, more preferably 5 to 200 m/s. Preferably, the drying air can be directed by one or more hollow cylinders.

In another preferred embodiment, the collector is covered with an inert material with a low surface energy, for example with polytetrafluoroethylene or polyethylene, from which the fibres are easily removed, or it is covered with a textile, for example polyester knitted fabric, on which the fibres are deposited and which remains a part of the final product.

Preferably, the fibres have a diameter of 100 nm to 100 μm and form a non-woven fabric on the collector, having an area weight 0.1 to 120 g/m².

In another embodiment, the spinning solution comprises hyaluronan 3-(2-furanyl) acryloyl ester, or a mixture of hyaluronic acid or a water-soluble salt thereof and hyaluronan 3-(2-furanyl) acryloyl ester, with the total concentration of the mixture of 1 to 10% by weight and the resulting fibres in the form of a non-woven fabric are subsequently crosslinked by radiation with a wavelength in the range from 280 to 750 nm, preferably 302 nm, for 2-60 minutes, wherein the substitution degree of hyaluronan with 3-(2-furanyl) acryloyl is in the range from 0.1 to 20%, and the proportion of hyaluronan 3-(2-furanyl) acryloyl ester in the mixture with the native HA is at least 0.1%, preferably 0.1-75%.

In any of the above-mentioned embodiments, the spinning solution can preferably contain another polymer, such as polyethylenoxide, polyvinylpyrrolidone, pullulan, carboxymethyl cellulose or oxycellulose, and/or one or more pharmaceutically and/or cosmetically acceptable low molecular weight substances that can be dissolved or dispersed in the solvent mixture used, selected from the group comprising antibacterial agents, e.g., octenidine dihydrochloride or carbethopendecinium bromide; antivirals, e.g., acyclovir; antifungals, e.g., clotrimazole or terbinafine; drugs, e.g., lidocaine hydrochloride; vitamins, e.g., riboflavin; plant extracts, e.g., bisabolol; surfactants, e.g., polysorbate 80; peptides, e.g., antimicrobial peptides, such as cathelicidine LL-37, pexiganan MSI-78, or WR-12; wound healing promoting peptides, e.g., dalargin, TP-508, or biotin-GHK; hormonal peptides, e.g., lysipressin or terlipressin; dyes, e.g., Patent Blue VF.

The method according to the present disclosure is carried out first by preparing a spinning solution of hyaluronic acid and/or a water-soluble metal or non-metal salt or a water-soluble mixture of metal and/or non-metal salts of hyaluronic acid and/or hyaluronic acid derivative containing 0.08 to 3% by weight of hyaluronic acid and/or a metal or non-metal salt thereof or a water-soluble mixture of metal and/or non-metal salts of hyaluronic acid and/or hyaluronic acid derivative, having a high molecular weight, 0.1 to 22% by weight of hyaluronic acid and/or a metal or non-metal salt thereof or a water-soluble mixture of metal and/or non-metal salts of hyaluronic acid and/or hyaluronic acid derivative, having a low molecular weight, 0 to 50% by weight of an organic solvent and 48 to 95% by weight of water, preferably 1 to 2% by weight of hyaluronic acid and/or a metal or non-metal salt thereof or a mixture of metal and/or non-metal salts of hyaluronic acid and/or a derivative thereof having a high molecular weight, 1 to 8% by weight of hyaluronic acid and/or a metal or non-metal salt thereof or a water-soluble mixture of metal and/or non-metal salts of hyaluronic acid and/or hyaluronic acid derivative, having a low molecular weight, 25 to 40% by weight of 2-propanol and 58 to 73% by weight of water.

The initial hyaluronic acid and/or the water-soluble metal or non-metal salt thereof and/or the derivative thereof having a high molecular weight has a weight average molecular weight (determined by the method of Size Exclusion Chromatography coupled to Multi-Angle Laser Light Scattering, SEC-MALLS) of 1.1 MDa to 2.5 MDa. The initial hyaluronic acid and/or the water-soluble metal or non-metal salt thereof and/or the derivative thereof having a high molecular weight has a weight average molecular weight (determined by the method of Size Exclusion Chromatography coupled to Multi-Angle Laser Light Scattering, SEC-MALLS) of 5 kDa to 750 kDa. The molecular weights used must be adapted to the concentration of the polymer in the spinning solution to achieve its optimal viscosity, for higher molecular weights lower concentrations must be chosen and vice versa. If the viscosity of the spinning solution is too high, there is no effective elongation of the resulting fibre and the extruded solution stream breaks instead.

In a preferred embodiment, the spinning solution comprises a solution of a hyaluronic acid derivative, which is hyaluronan chloramide, or a mixture of a hyaluronic acid derivative, which is hyaluronan chloramide, and native hyaluronic acid, wherein the substitution degree of hyaluronan chloramide is in the range from 0.1% to 100%, preferably 50 to 100%.

The procedure for preparing the spinning solution is preferably such that first hyaluronic acid and/or the metal or non-metal salt thereof or the water-soluble mixture of metal and/or non-metal salts of hyaluronic acid and/or hyaluronic acid derivative are dispersed in an organic solvent, then water is added with thorough mixing, or optionally, they are dispersed in water directly, and the solution is stirred for 1 to 24 hours at the temperature 20 to 30° C. until the polymer is completely dissolved. The spinning solution is then filled into the cartridge of a pneumatic dosing device (working on the principle of extruding the solution from the storage cartridge with compressed air, an example of such a dosing device is the Vieweg DC 1200), the cartridge is sealed and connected to the compressed air with an overpressure of +5 to +7 bar for 1 to 8 hours until the gas bubbles dissolve.

The spinning solution is extruded by an extrusion piece with at least one dispensing opening with an internal diameter of 80 to 410 micrometers, preferably 100 to 260 micrometers. The term extrusion piece in this description preferably refers to a conventional extrusion needle with a blunt end and with 1 to 4 capillaries, which may be straight or bent to allow their better positioning in the air stream, or a conical extrusion needle, the internal diameter of which gradually decreases up to the dispensing opening, thanks to which it can dispense viscous solutions at a lower pressure, or a nozzle containing more than one dispensing opening, preferably more than 10, more preferably more than 50 dispensing openings arranged for example in a row.

The spinning solution is extruded from the dispensing opening with a rate from 0.01 to 0.7 mL/min. At a lower dosing rate, drawing and air-drying of the fibres are both more efficient and the fibres fall onto the collector dried better, but at the same time the spinning solution in the extrusion piece is more heated and especially when the extrusion piece is flown around with air of a higher temperature, there is a risk of drying the solution and clogging the dispensing opening.

The spinning solution is extruded from the dispensing opening into a stream of drying air that flows around the extrusion piece. The stream of drying air draws the extruded spinning solution into a fibre shape, speeds up the evaporation of the solvent from the spinning solution and drifts the resulting fibres onto the collector.

In a preferred embodiment, the extrusion piece is flown around by a smaller amount of cooler air, which carries the spinning solution into a stream of a larger amount of warmer drying air, wherein this embodiment prevents overheating of the spinning solution already in the extrusion piece.

The temperature of the drying air is 15 to 250° C., preferably 15 to 160° C., the absolute humidity of the drying air is 0 to 14 g/m³, preferably 0 to 2 g/m³, the drying air flow rate is 1.6 to 315 m/s, preferably 5 to 200 m/s. The air flow rate at the nozzle outlet depends on the combination of the nozzle cross-section and the volumetric flow rate. In general, when using a higher drying air flow rate, a lower drying air temperature can be used to effectively dry the fibres and vice versa, because the faster flowing drying air draws the resulting fibre more effectively into a smaller diameter fibre from which the drying air more easily removes solvents. Similarly, the use of a dispensing opening with a smaller internal diameter supports the formation of a fibre with a smaller diameter.

In a preferred embodiment, the drying air carrying the resulting fibre is directed by one or more hollow cylinders, which prevent the expansion of the drying air stream and its mixing with the surrounding air, thereby reducing its speed and temperature.

The fibres are deposited in the form of a non-woven fabric on a collector with a shape of a cylinder, bars, belt or board. In one preferred embodiment, the collector is in the form of an inverted cone disposed in a cylindrical vessel, wherein the side edges of the cone are formed by bars. The collector is immobile or preferably mobile, such as a cylinder rotating around its axis or a belt rewound from cylinder to cylinder. The surface of the collector is preferably covered with an inert material with a low surface energy, for example with polytetrafluoroethylene or polyethylene, from which the fibres can be easily removed. The collector's distance from the dispensing opening is preferably in the range from 5 cm to 140 cm. In another embodiment, the collector is covered with a fabric or a film, for example polyester or polyamide fabric or film, which forms one of the layers of the final product, and the prepared fibres are applied directly thereon as another layer.

In a preferred embodiment the arrangement of the collector is such that the fibres deposited on the collector hang most of their length in the air and can thus dry more effectively. In this embodiment, the collector is formed for example by bars or is covered with a fabric with larger meshes.

In another preferred embodiment the collector with the shape of a cylinder rotating around its axis can be moved along this axis, so that the fibres gradually fall on its entire surface in a uniformly thick layer.

In another preferred embodiment the collector is an object on the surface of which a layer of a non-woven fabric is formed and then together with the object it forms the final product. The collector is for example an implantable medical device (e.g. pacemaker or joint) on the surface of which a bio-compatibilizing or an antimicrobial layer is formed, for example on the basis of hyaluronan chloramide, or on the basis of hyaluronan, with an addition of octenidine dihydrochloride.

Further, the disclosure provides a two-dimensional or three-dimensional material made of microfibres or nanofibres prepared in the manner described above. The non-woven fabric as contemplated herein has preferably an area weight from 0.1 to 120 g/m² and is made of fibres with a diameter in the range from 100 nm to 100 micrometers. Non-woven fabrics of higher area weights, approx. from 5 g/m², can be used as self-supporting two-dimensional materials. Non-woven fabrics of lower area weights are not rigid enough to maintain the shape of a flat structure, and after being removed from the collector, they self-fold into a three-dimensional structure similar to cotton wool.

As mentioned above, the proportion of solvents contained in the fibres when falling on the collector can be influenced by the parameters selection (in particular, the rate of spinning solution dosing, the diameter of the dispensing opening, the drying air temperature and the drying air flow rate). For obtaining high-quality fibres and the non-woven fabric formed by them, preferably such combination of parameters is selected, that the fibres fall on the collector dry enough (i.e., with such a low residual proportion of solvents) to retain the shape of the fibre and to complete drying quickly enough (preferably within a few tens of seconds at most) to prevent a layer of gel growing on the collector. The proportion of solvents with respect to the polymer may also be reduced by adding a low-molecular weight polymer to the spinning solution, which significantly increases the overall polymer concentration without significantly changing the viscosity of the spinning solution. If not completely dried fibres fall on each other and coalesce in the places where they cross each other. In a preferred embodiment, the fibres fall on the collector dry enough, so their coalescence does not occur. The resulting non-woven fabric is exemplified by a lower volumetric mass. In another preferred embodiment, the fibres fall on the collector dry enough to directly form a three-dimensional, cotton wool-like formation, so it is not necessary to assemble such a formation from thin two-dimensional layers.

Furthermore, the present disclosure provides a device for the production of two-dimensional or three-dimensional materials from microfibres or nanofibres, which includes an extrusion piece containing a pass-through channel, which has an inlet opening for feeding the spinning solution and at least one dispensing opening for dispensing the spinning solution. Furthermore, the device comprises an air nozzle, the main air outlet opening of which is arranged to direct the exiting air to an area surrounding the dispensing opening of the extrusion piece parallel to the axis of the dispensing opening of the extrusion piece, the air nozzle comprising additional outlet openings arranged to direct the additional air flow to the area below the dispensing opening into the air flow from the main air outlet opening at an angle of 30° to 60°, preferably 40° to 50°, more preferably 45°. The device further comprises at least one cooling channel for guiding the coolant liquid through the walls of the air nozzle.

In a preferred embodiment the device contains a source of drying air, air nozzle, device for dosing of the spinning solution, extrusion piece, e.g., a extrusion needle, positioned coaxially with the air nozzle, and a collector which is arranged at a distance from the extrusion piece and the dispensing opening of the extrusion piece is facing the collecting surface of the collector.

The collector may be, for example, in the form of a cylinder, a cylinder formed by bars, an inverted cone formed by bars, or in the form of an implantable medical device.

In a preferred embodiment, the device comprises a heating element for heating the air supplied to the additional outlet openings of the air nozzle.

In another preferred embodiment, the stream of the drying air carrying the stream of the spinning solution is directed through a defined space, which prevents mixing with the surrounding air, by employing a focusing part arranged between the collector and the dispensing opening of the extrusion piece and containing a pass-through cavity, the axis of which is identical to the axis of the dispensing opening. The main outlet opening of the air nozzle is arranged coaxially with the dispensing opening of the extrusion piece and is preferably annular or circular, wherein the extrusion piece area containing the dispensing opening is arranged in the area of the main outlet opening of the air nozzle, and/or the main outlet opening of the air nozzle is arranged further away from the collector than the dispensing opening.

The prepared two-dimensional and three-dimensional fibrous materials can be used in medicine as covers or fillings for wound treatment, or as envelopes for implantable medical devices.

The device provided herein thus comprises an extrusion piece 11 comprising a pass-through channel having an inlet opening for feeding a spinning solution 9 and a dispensing opening 11a for discharging of the spinning solution, and an air nozzle 5, having a main outlet opening 4 for the exit of air which is arranged for directing of the exiting air into the region surrounding the dispensing opening 11a of the extrusion piece 11 in parallel to the axis of the dispensing opening 11a of the extrusion piece 11. Furthermore, the device preferably comprises a collector 14 arranged in a certain distance from the extrusion piece 11, wherein the dispensing opening 11a of the extrusion piece 11 faces the collecting surface of the collector 14.

In a preferred embodiment, the device comprises a focusing part 13 arranged between the collector 14 and the dispensing opening 11a of the extrusion piece 11, said focusing part 13 comprising a pass-through cavity, the axis of which being identical to the axis of the dispensing opening 11a.

The main outlet opening 4 of the air nozzle 5 is preferably annular and arranged coaxially with the dispensing opening 11a of the extrusion piece 11 and/or the main outlet opening 4 of the air nozzle 5 is arranged farther from the collector 14 than the dispensing opening 11a.

The device may comprise a compressor 1 for feeding the air into the air nozzle 5.

In another preferred embodiment, the device also comprises a heating element 2 for heating the air fed into the air nozzle 5.

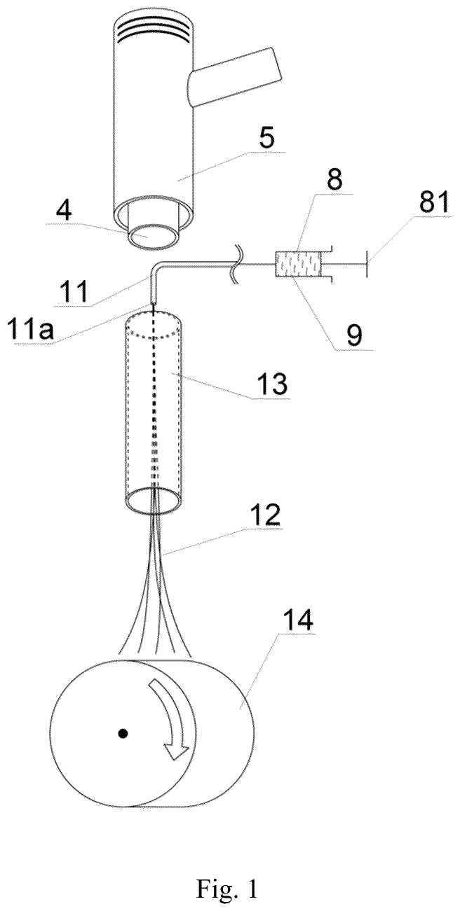

FIG. 1 shows schematically an embodiment of the device for the production of two-dimensional or three-dimensional fibre materials from microfibres or nanofibres, wherein the device comprises an extrusion piece 11 in the form of an extrusion needle through which a pass-through channel extends, said pass-through channel comprising a feeding opening and a dispensing opening 11a. The extrusion piece 11 is adapted for a fluid connection with a dosing device 8 on the side of the feeding opening, in this case in the form of a cartouche with a piston 81.

The pass-through channel has a diameter of 80 to 410 micrometres, preferably 100 to 210 micrometres, at least in the region of the dispensing opening 11a.

The device further comprises an air nozzle 5, here in the form of a hot-air pistol. The air nozzle 5 has a main outlet opening 4 which is directed and adapted for directing the hot air stream such that it flows around the extrusion piece 11 in parallel to the direction of the exit of the spun solution from the dispensing opening 11a of the extrusion piece 11. To this end, the air nozzle 5 is arranged so that the axis of its main outlet opening 4 corresponds to the axis of the dispensing opening 11a of the extrusion piece 11.

The device further comprises a collector 14 for depositing the produced fibres, in this case in the form of a rotatably arranged cylinder, wherein the dispensing opening 11a of the extrusion piece 11 faces the depositing surface of the collector 14.

A focusing part 13 having a pass-through cavity is arranged between the collector 14 and the dispensing opening 11a of the extrusion piece 11, wherein the axis of said pass-through cavity corresponds to the axis of the dispensing opening 11a. The pass-through cavity of the focusing part 13 is cylindrical.

By employing the piston 81, a polymer solution 9 is extruded from the dosing device 8 by employing a bent extrusion needle forming an extrusion piece 11 and being arranged coaxially under the main outlet opening 4 of the air nozzle 5. The resulting fibres are led through the focusing part 13 onto the collector 14.

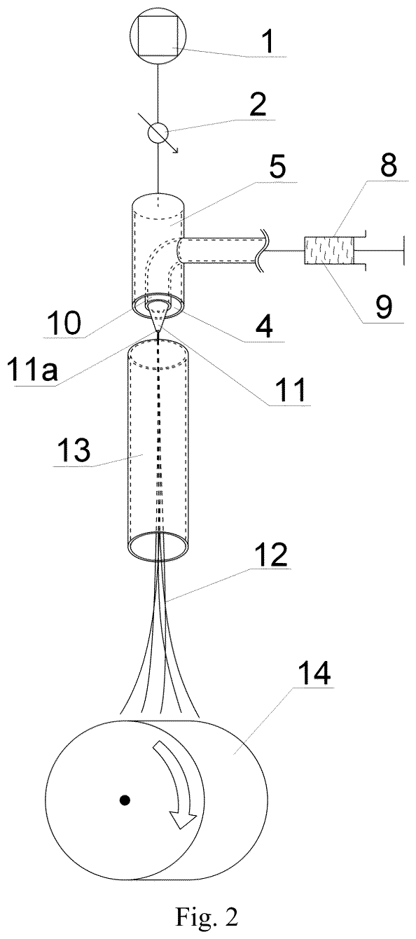

FIG. 2 shows schematically an embodiment of the device for the production of two-dimensional or three-dimensional fibre materials from microfibres or nanofibres which differs from the device of FIG. 1 particularly in that the extrusion piece 11 is partially housed inside the air nozzle 5 and at the same time protrudes from the air nozzle 5 with the part that comprises the dispensing opening 11a. Furthermore, the device of FIG. 2 is supplemented with a compressor 1 which is fluidly interconnected with the air nozzle 5 for feeding an air stream to the air nozzle 5. A heating element 2 for heating the air fed to the air nozzle 5 is arranged in a piping interconnecting the compressor 1 and the air nozzle 5.

The polymer solution 9 is brought from the dosing device 8 to the extrusion piece 11 which comprises a tube 10 followed by an extrusion needle. The air stream generated by the compressor 1 is heated by the heating element 2 and exits the air nozzle 5 through the main outlet opening 4 having a shape of annulus, the dispensing opening 11a of the extrusion piece 11, or extrusion needle, being coaxially arranged therewith. The resulting fibres 12 are led by the focusing cylinder 13 onto the collector 14.

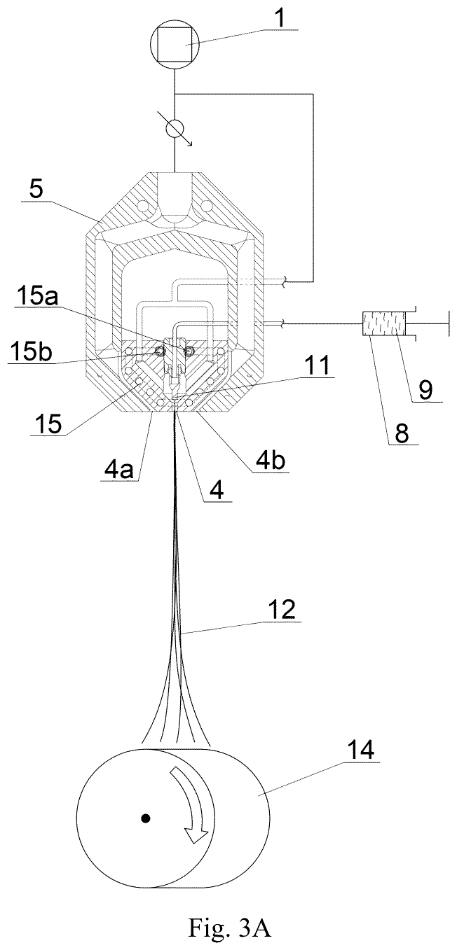

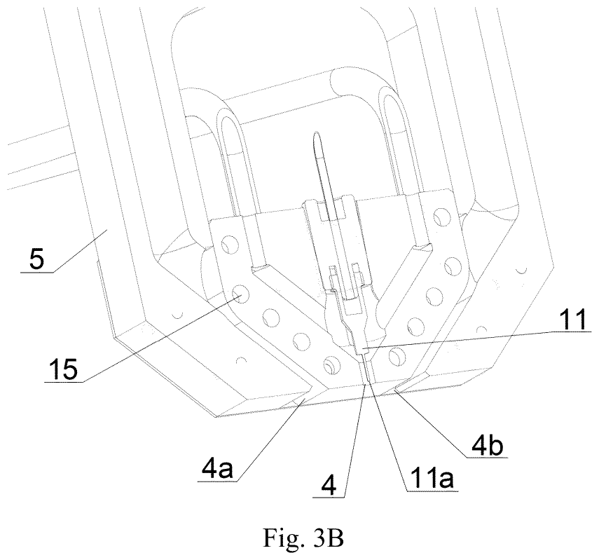

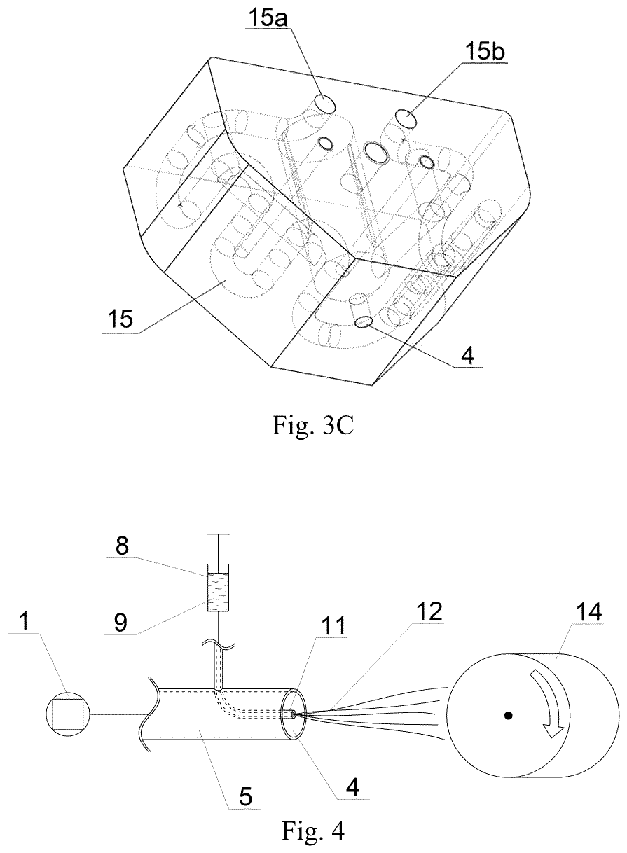

FIGS. 3A, 3B, and 3C show schematically an embodiment of the device for the production of two-dimensional or three-dimensional fibre materials from microfibres or nanofibres as contemplated herein with a combination of non-heated and heated air.

The device of FIGS. 3A, 3B, and 3C differs from the device of FIG. 2 particularly in that the dispensing opening 11a of the extrusion piece 11 is arranged substantially in the same plane as the main outlet opening 4 of the air nozzle 5 and that additional outlet openings 4a, 4b of additional air nozzle 5 are directed to the region below the dispensing opening 11a of the extrusion piece.

The axes of the additional outlet openings 4a, 4b of the air nozzle 5 intersect the axis of the dispensing opening 11a of the extrusion piece 11 and form an angle of 45° with said axis.

FIG. 3C shows that the air nozzle 5 comprises a cooling channel 15 for a coolant liquid for cooling the air nozzle 5, wherein said cooling channel 15 has an inlet opening 15a adapted for connecting a hose for feeding the coolant liquid and an outlet opening 15b adapted for connecting a hose for discharging the coolant liquid. Between the inlet opening 15a and the outlet opening 15b, the cooling channel 15 passes in a labyrinth-like manner through the material of the wall of the air nozzle 5 for effective cooling of the air nozzle 5.

The dosing device 8 extrudes the polymer solution 9 through a straight extrusion needle forming the extrusion piece 11 arranged coaxially in the main outlet opening 4 of the air nozzle 5, into which the air from the compressor 1 is fed. Said air brings the solution into a flow of warmer (heated) air exiting the outlet openings 4a, 4b. The coolant flows inside the channel 15, which prevents the heat transfer from the heated air to the non-heated air through the body of the nozzle 5. The resulting fibres 12 are led onto the collector 14.

FIG. 4 shows schematically an embodiment of the device for the production of two-dimensional or three-dimensional fibre material as contemplated herein based on the solution blow spinning technology with a compressor 1 as a source of air and a narrow air nozzle 5 through which the air exits with a high velocity. The diameter of the air nozzle 5 is in the range of 2 to 4 mm, and the velocity of the air is in the range of 50 to 350 m/s. The diameter of the needle is in the range of 80 to 210 micrometres. A dosing device 8 extrudes a polymer solution 9 through a bent extrusion needle forming an extrusion piece 11 arranged coaxially in the outlet opening 4 of the air nozzle 5. The resulting fibres 12 are deposited on the collector 14.

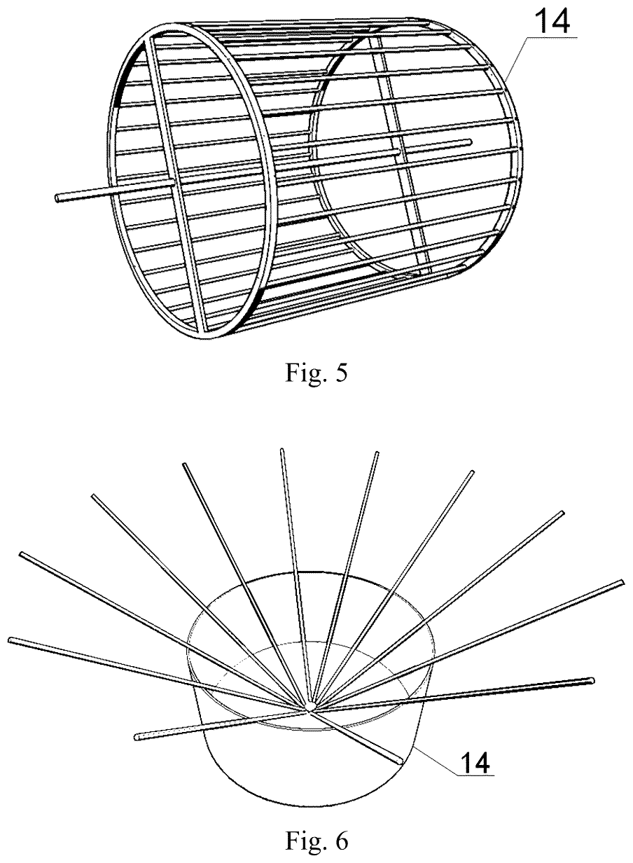

FIG. 5 shows an example of the collector 14 in the form of a rotatably arranged cylindrical cage.

FIG. 6 shows an example of the collector 14 in form of a stationary conical cage.

### LIST OF THE REFERENCE SIGNS IN THE DRAWINGS

1 compressor
2 heating element
4 main outlet opening of the air nozzle
4a, 4b additional outlet openings of the air nozzle
5 air nozzle
8 dosing device
81 piston
9 spinning solution
10 tube
11 extrusion piece
11a dispensing opening
12 fibers
13 focusing part
14 collector
15 cooling liquid channel
15a inlet opening
15b outlet opening

### EXAMPLES

The following examples demonstrate various embodiments of the method according to the present disclosure. The air velocity is primarily indicated in l/min, but in some examples it is converted to an approximate air velocity in m/s at the exit of the air nozzle 5.

The terms “hyaluronic acid”, “hyaluronan” and “native hyaluronic acid” always refer to hyaluronic acid, the sodium salt thereof and the mixture of the acid and the sodium salt, unless otherwise indicated.

### Example 1

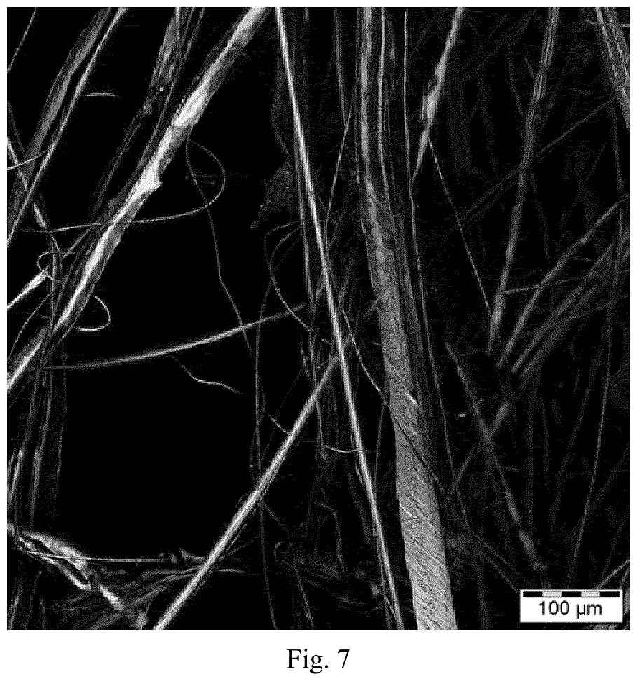

2 grams of sodium hyaluronan having the weight average molecular weight 2.12 MDa and 2 grams of sodium hyaluronan having the weight average molecular weight 85 kDa are dispersed in 52.5 mL of 2-propanol. 61.9 mL of water are added to the resulting dispersion with thorough mixing, the solution is stirred for 24 hours at 21° C. until the polymer is completely dissolved, resulting in a solution having the concentration 1.87% by weight of sodium hyaluronan having the weight average molecular weight 2.12 MDa and 1.87% by weight of sodium hyaluronan having the weight average molecular weight 85 kDa. The spinning solution 9 is then filled into the cartridge of the pneumatic dosing device 8, the cartridge is sealed and connected to the compressed air of +6 bar for 5 hours until the gas bubbles dissolve. Subsequently, the spinning solution 9 is spun on the device shown in FIG. 1. The spinning solution 9 is extruded by the extrusion part 11, which is a bent blunt extrusion needle having the length of 12.7 mm with an internal diameter of 210 micrometers with the rate of 0.241 mL/min. The extrusion needle is located coaxially below the air nozzle 5, which is formed by a point nozzle of the heat gun Wagner FURNO 750 so that the end of the extrusion needle is located 15 mm below the main outlet opening 4 of the air nozzle 5. The air nozzle 5 blows the drying air of the temperature of 160° C. and absolute humidity of 7 g/m³ with the volume flow of 220 L/min, which corresponds to the average air velocity of 13 m/s. The resulting fibers 12 are carried by the air stream to the collector 14 in the shape of a cylinder with a diameter of 15 cm formed by 38 bars of 3 mm thickness (shown in FIG. 5) rotating at the speed of 2 revolutions per minute located 80 cm below the extrusion needle. The surface of the collector 14 is covered with a polytetrafluoroethylene foil. The resulting fibers 12 having the diameter of 1.5-40 micrometers were obtained from the collector 14 in the form of thin layers that were packed into a bulky 3D structure (cotton wool) (FIG. 7).

### Example 2

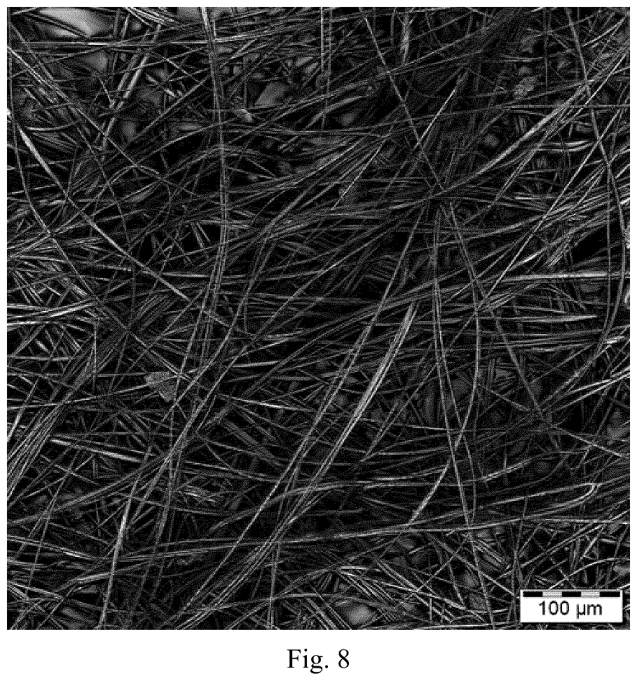

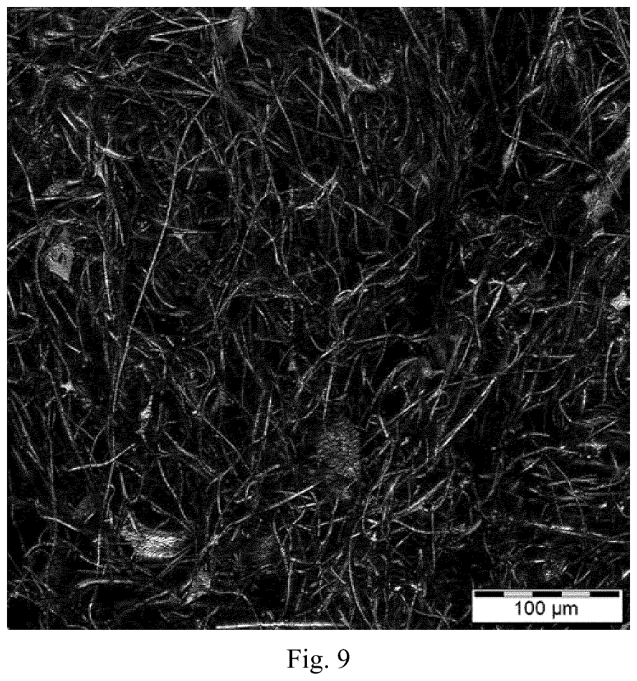

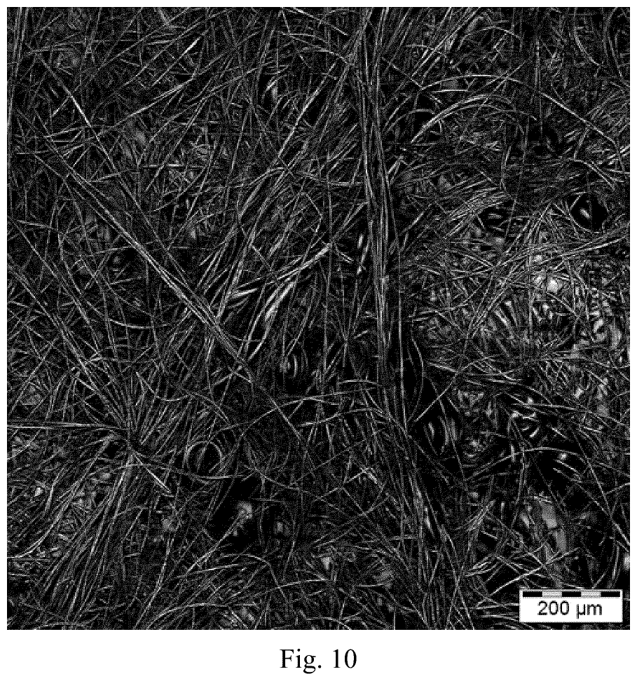

2 grams of sodium hyaluronan having the weight average molecular weight 2.12 MDa and 2 grams of sodium hyaluronan having the weight average molecular weight 85 kDa are dispersed in 52.5 mL of 2-propanol. 61.9 mL of water are added to the resulting dispersion with thorough mixing, the solution is stirred for 18 hours at 20° C. until the polymer is completely dissolved, resulting in a solution having the concentration 1.87% by weight of sodium hyaluronan having the weight average molecular weight 2.12 MDa and 1.87% by weight of sodium hyaluronan having the weight average molecular weight 85 kDa. The spinning solution 9 is then filled into the cartridge of the pneumatic dosing device 8, the cartridge is sealed and connected to the compressed air of +5.0 bar for 7 hours until the gas bubbles dissolve. Subsequently, the spinning solution 9 is spun on the device shown in FIG. 2. The spinning solution 9 is extruded by the extrusion part 11, which is a blunt extrusion needle of the length of 6.4 mm with an internal diameter of 160 micrometers with the rate of 0.2 mL/min. The extrusion needle is located coaxially in the center of the air nozzle 5, the end of the needle is located 20 mm below the end of the air nozzle 5. The drying air is flowing from the air nozzle 5 through an annular outlet opening 4 with an internal diameter of 19 mm and external diameter 25 mm, the temperature of the drying air is 100° C., its absolute humidity is 0.2 g/m³ and the volume flow is 300 L/min, corresponding to the average air velocity of 24 m/s. The resulting fibers 12 are carried by the air stream to the stationary collector 14 (shown in FIG. 6) in the shape of an inverted cone located in a cylindrical container with the height of 12 cm and an internal diameter of 26 cm, the side edges of the cone being formed by 12 bars 34 cm long and 2 mm thick, and the opposite side edges forming an angle of 90°. The bottom of the cylindrical container, which is a part of the collector 14, is located 112 cm below the extrusion needle. The surface of the collector 14 is covered with a polytetrafluoroethylene foil. The resulting fibers 12 having the diameter of 1-6.5 micrometers on the collector bars form directly a bulky 3D structure (cotton wool) (FIG. 8).

### Example 3

2 grams of sodium hyaluronan having the weight average molecular weight of 2.12 MDa and 2 grams of sodium hyaluronan having the weight average molecular weight of 85 kDa are dispersed in 52.5 mL of 2-propanol. 61.9 mL of water are added to the resulting dispersion with thorough mixing, the solution is stirred for 20 hours at 24° C. until the polymer is completely dissolved resulting in a solution having the concentration 1.87% by weight of sodium hyaluronan having the weight average molecular weight 2.12 MDa and 1.87% by weight of sodium hyaluronan having the weight average molecular weight 85 kDa. The spinning solution 9 is then filled into the cartridge of the pneumatic dosing device 8, the cartridge is sealed and connected to the compressed air of +7.0 bar for 4 hours until the gas bubbles dissolve. Subsequently, the spinning solution 9 is spun on the device shown in FIG. 3A. The spinning solution 9 is extruded by the extrusion part 11, which is a blunt extrusion needle of the length of 6.4 mm with an internal diameter of 160 micrometers with the rate of 0.16 mL/min. The extrusion needle is located coaxially in the main outlet opening 4 of the air nozzle 5 with diameter 3 mm, the end of the needle is located 1 mm above the underside of the nozzle body 5. Through this main outlet opening 4 the air flows with the temperature of 30° C., absolute humidity of 0.2 g/m³ and with the volume flow of 30 L/min, corresponding to the average air velocity of 71 m/s. This air carries the stream of the spinning solution 9 to the stream of the heated air with the temperature of 147° C. and absolute humidity of 0.2 g/m³ with the volume flow of 200 L/min, corresponding to the average air velocity of 28 m/s, coming out simultaneously from two additional openings 4a, 4b of the nozzle 5, forming a 90° angle with each other and having a rectangular cross-section with sides of 2 and 30 mm and being arranged 20 mm from each other. Water having the temperature of 10° C. flows through the cooling channel 15 for cooling liquid having the diameter of 4 mm, with the volume flow of 250 mL/min. The resulting fibers 12 are carried by the air stream to the stationary collector 14 (shown in FIG. 6) in the shape of an inverted cone located in a cylindrical container with the height of 12 cm and an internal diameter of 26 cm, the side edges of the cone being formed by 12 bars 34 cm long and 2 mm thick, and the opposite side edges forming an angle of 90°. The bottom of the cylindrical container, which is a part of the collector 14, is located 124 cm below the extrusion needle. The surface of the collector 14 is covered with a polytetrafluoroethylene foil. The resulting fibers 12 having the diameter of 1-6.5 micrometers on the collector 14 bars form directly a bulky 3D structure (cotton wool).

### Example 4

1 gram of sodium hyaluronan having the weight average molecular weight 2.12 MDa and 2 grams of sodium hyaluronan having the weight average molecular weight 30 kDa are dispersed in 39.4 mL of 2-propanol. 46.4 mL of water are added to the resulting dispersion with thorough mixing, the solution is stirred for 16 hours at 22° C. until the polymer is completely dissolved resulting in a spinning solution 9 with the concentration of 1.25% by weight of sodium hyaluronan having the weight average molecular weight 2.12 MDa and 2.49% by weight of sodium hyaluronan having the weight average molecular weight 30 kDa. The spinning solution 9 is then filled into the plastic dosing device 8, which is a syringe that is inserted into a syringe pump. Subsequently, the spinning solution 9 is spun on the device shown in FIG. 4. The spinning solution 9 is extruded by the extrusion part 11, which is a bent blunt extrusion needle having a length of 12.7 mm and an internal diameter of 160 micrometers, at the rate of 0.01 mL/min. The extrusion needle is located coaxially in the air nozzle 5 formed by a polyamide tube of the internal diameter of 4 mm so that the end of the needle exceeds the tube by 1 mm. The air nozzle 5 blows the drying air having the temperature of 29° C. and absolute humidity of 0.2 g/m³ with the volume flow of 60 L/min, corresponding to the average air velocity of 80 m/s. The resulting fibers 12 are carried by the air stream to the collector 14 in the shape of a cylinder with a diameter of 7 cm rotating at the speed of 40 revolutions per minute and located at a distance of 10 cm from the extrusion needle. The surface of the collector 14 is covered with a low-density polyethylene foil. The resulting fibers 12 having the diameter of 0.25-3.5 micrometers were removed from the collector 14 in the form of thin layers that were packed into a 3D structure (cotton wool) (FIG. 9).

### Example 5

The procedure was the same as in Example 2, only with the differences, that the spinning solution 9 containing 2.85% by weight of sodium hyaluronan having the weight average molecular weight 1.17 MDa and 2.85% by weight of sodium hyaluronan having the weight average molecular weight 85 kDa was prepared from 3 grams of sodium hyaluronan having the weight average molecular weight 1.17 MDa, 3 grams of sodium hyaluronan having the weight average molecular weight 85 kDa, 50.7 mL of 2-propanol, and 59.7 mL of water. The spinning solution 9 was extruded through an extrusion needle having an internal diameter of 210 micrometers with the rate of 0.24 mL/min. The air nozzle 5 blew the drying air with the volume flow of 400 L/min, which corresponds to the average air velocity of 32 m/s. The resulting fibers 12 having a diameter of 2-13 micrometers were obtained from the collector 14 in the form of a porous non-woven textile.

### Example 6

The procedure was the same as in Example 3, only with the differences, that the spinning solution 9 containing 0.40% by weight of sodium hyaluronan having the weight average molecular weight 2.12 MDa and 6.54% by weight of sodium hyaluronan having the weight average molecular weight 85 kDa was prepared from 0.4 grams of sodium hyaluronan having the weight average molecular weight 2.12 MDa, 6.6 grams of sodium hyaluronan having the weight average molecular weight 85 kDa, 41.9 mL of 2-propanol, and 61.1 mL of water. The spinning solution 9 was extruded with the rate of 0.08 mL/min. The resulting fibers 12 having a diameter of 0.6-2.2 micrometers were obtained from the collector 14 in the form of a bulky 3D structure (cotton wool).

### Example 7

The procedure was the same as in Example 3, only with the differences, that the spinning solution 9 containing 1.89% by weight of sodium hyaluronan having the weight average molecular weight of 2.05 MDa and 0.94% by weight of sodium hyaluronan having the weight average molecular weight 730 kDa was prepared from 2 grams of sodium hyaluronan having the weight average molecular weight of 2.05 MDa, 1 gram of hyaluronan having the weight average molecular weight 730 kDa, 52.5 mL of 2-propanol, and 61.9 mL of water. The spinning solution 9 was extruded with the rate of 0.08 mL/min. The resulting fibers 12 having a diameter of 0.5-7 micrometers were obtained from the collector 14 in the form of a bulky 3D structure (cotton wool).

### Example 8

The procedure was the same as in Example 3, only with the differences, that the spinning solution 9 containing 1.58% by weight of sodium hyaluronan having the weight average molecular weight 2.12 MDa and 10.9% by weight of sodium hyaluronan having the weight average molecular weight 30 kDa was prepared from 1.8 grams of sodium hyaluronan having the weight average molecular weight 2.12 MDa, 12.5 grams of sodium hyaluronan having the weight average molecular weight 30 kDa, and 100 mL of water. The spinning solution 9 was extruded at a rate of 0.08 mL/min. The resulting fibers 12 having a diameter of 0.8-12 micrometers were obtained from the collector 14 in the form of a bulky 3D structure (cotton wool).

### Example 9

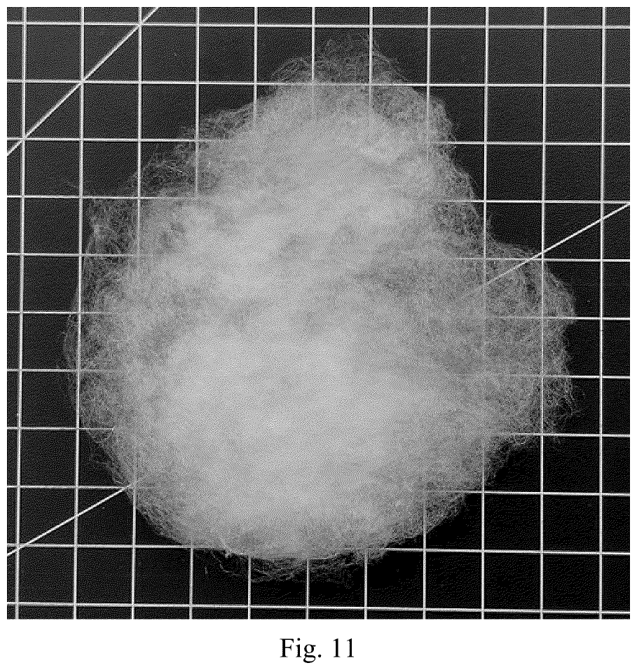

The procedure was the same as in Example 2, only with the differences, that the spinning solution 9 containing 1.67% by weight of sodium hyaluronan having the weight average molecular weight 2.12 MDa and 9.28% by weight of sodium hyaluronan having the weight average molecular weight 30 kDa was prepared from 2 grams of sodium hyaluronan having the weight average molecular weight 2.12 MDa, 11.1 grams of sodium hyaluronan having the weight average molecular weight 30 kDa, 34.0 mL of 2-propanol, and 80.0 mL of water. The spinning solution 9 was extruded by the extrusion piece 11, which was a conical extrusion needle Tecdia ARQUE-S having an internal diameter of 100 micrometers. The drying air was directed by a cylinder 13 located coaxially with the extrusion needle, the cylinder 13 of the internal diameter 6.7 cm and length 14 cm starting at 1 cm below the end of the extrusion needle. The air nozzle 5 blew the drying air with the volume rate of 400 L/min, which corresponds to the average air velocity of 32 m/s. The resulting fibers 12 having a diameter of 3-20 micrometers were obtained from the collector 14 in the form of a bulky 3D structure (cotton wool) (FIG. 11).

### Example 10

The procedure was the same as in Example 2, only with the differences, that the spinning solution 9 containing 2.93% by weight of sodium hyaluronan having the weight average molecular weight 1.17 MDa and 0.105% by weight of sodium hyaluronan having the weight average molecular weight 30 kDa was prepared from 3 grams of sodium hyaluronan having the weight average molecular weight 1.17 MDa, 107 milligrams of sodium hyaluronan having the weight average molecular weight 30 kDa, 50.7 mL of 2-propanol, and 59.7 mL of water. The air nozzle 5 blew the drying air with the volume flow of 400 L/min, which corresponds to the average air velocity of 32 m/s. The resulting fibers 12 having a diameter of 0.5-6.5 micrometers were obtained from the collector 14 in the form of a porous non-woven textile.

### Example 11

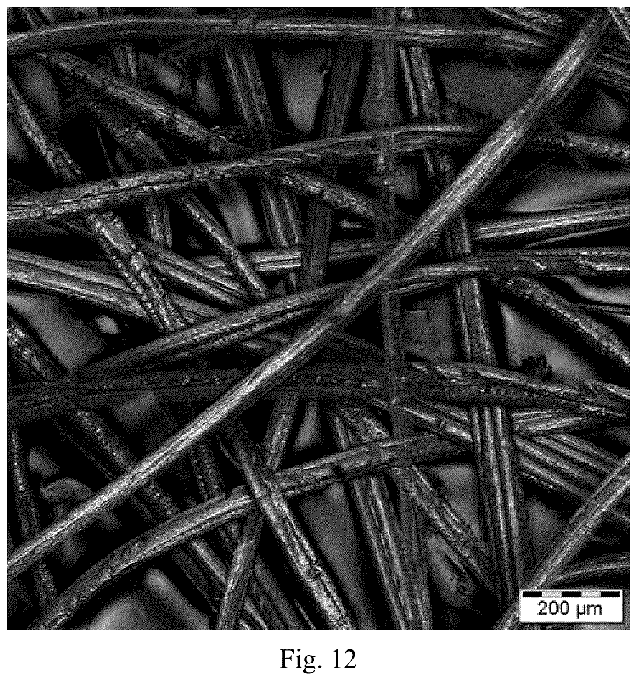

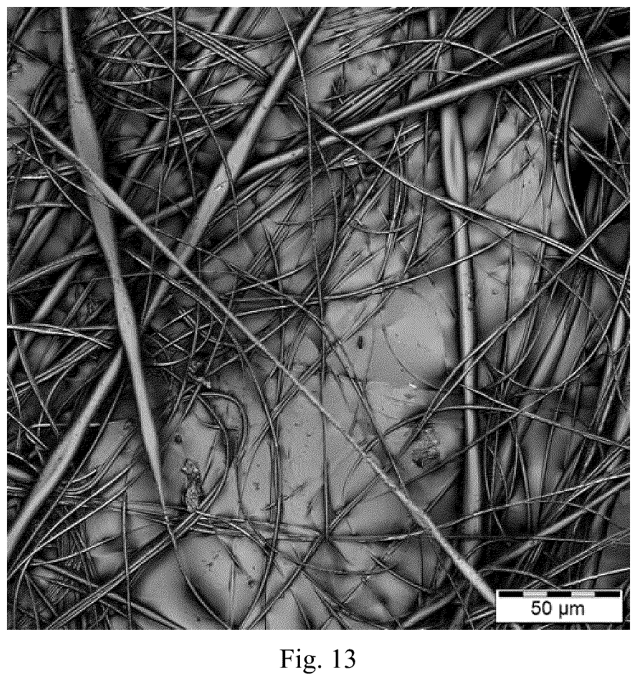

The procedure was the same as in Example 3, only with the differences, that the spinning solution 9 containing 0.081% by weight of sodium hyaluronan having the weight average molecular weight 1.17 MDa and 20.3% by weight of sodium hyaluronan having the weight average molecular weight 30 kDa, and 1.63% by weight of polyethylene oxide having the viscosity average molecular weight 1 MDa was prepared from 0.1 grams of sodium hyaluronan having the weight average molecular weight 1.17 MDa, 25 grams of sodium hyaluronan having the weight average molecular weight 30 kDa, 2 grams of polyethylene oxide, 72 mL of water, and 30.6 mL of 2-propanol. The spinning solution 9 was extruded by the extrusion piece 11, which was an extrusion needle having an internal diameter of 340 micrometers. The resulting fibers 12 having a diameter of 18-90 micrometers were obtained from the collector 14 in the form of a bulky 3D structure (cotton wool) (FIG. 12).

### Example 12

The procedure was the same as in Example 3, only with the differences, that the spinning solution 9 containing 1.71% by weight of sodium hyaluronan having the weight average molecular weight 1.86 MDa, 1.71% by weight of sodium hyaluronan having the weight average molecular weight 76 kDa, and 0.0137% by weight of octenidine dihydrochloride was prepared from 1.5 grams of sodium hyaluronan having the weight average molecular weight 1.86 MDa, 1.5 grams of sodium hyaluronan having the weight average molecular weight 76 kDa, 0.012 grams of octenidine dihydrochloride, 53.6 mL of ethanol, and 42.3 mL of water. The resulting fibers 12 having a diameter of 1-13 micrometers were removed from the collector 14 in the form of a bulky 3D structure (cotton wool).

### Example 13

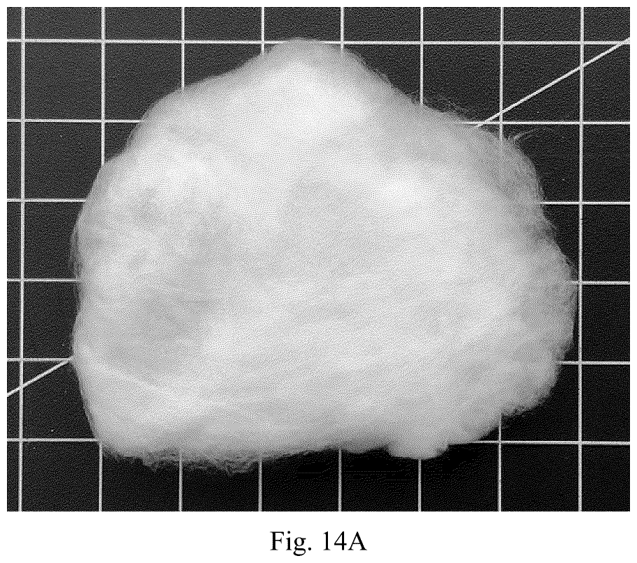

The procedure was the same as in Example 3, only with the differences, that the spinning solution 9 containing 1.83% by weight of sodium hyaluronan having the weight average molecular weight 2.05 MDa and 2.74% by weight of sodium hyaluronan having the weight average molecular weight 76 kDa was prepared from 2 grams of sodium hyaluronan having the weight average molecular weight 2.05 MDa, 3 grams of sodium hyaluronan having the weight average molecular weight 76 kDa, 46.3 mL of acetone, and 67.9 mL of water. The spinning solution 9 was extruded with the rate of 0.08 m¹/min. The resulting fibers 12 having a diameter of 0.2-10 micrometers were obtained from the collector 14 in the form of a bulky 3D structure (cotton wool) (FIG. 13).

### Example 14

The procedure was the same as in Example 3, only with the differences, that the spinning solution 9 containing 1.67% by weight of sodium hyaluronan having the weight average molecular weight 2.05 MDa and 9.28% by weight of sodium hyaluronan having the weight average molecular weight 30 kDa was prepared from 2 grams of sodium hyaluronan having the weight average molecular weight 2.05 MDa, 11.1 grams of sodium hyaluronan having the weight average molecular weight 30 kDa, 34 mL of 2-propanol, and 80 mL of water. The spinning solution 9 was extruded with the rate of 0.12 m¹/min. The resulting fibers 12 having a diameter of 2.5-20 micrometers were obtained from the collector 14 in the form of a bulky 3D structure (cotton wool).

### Example 15

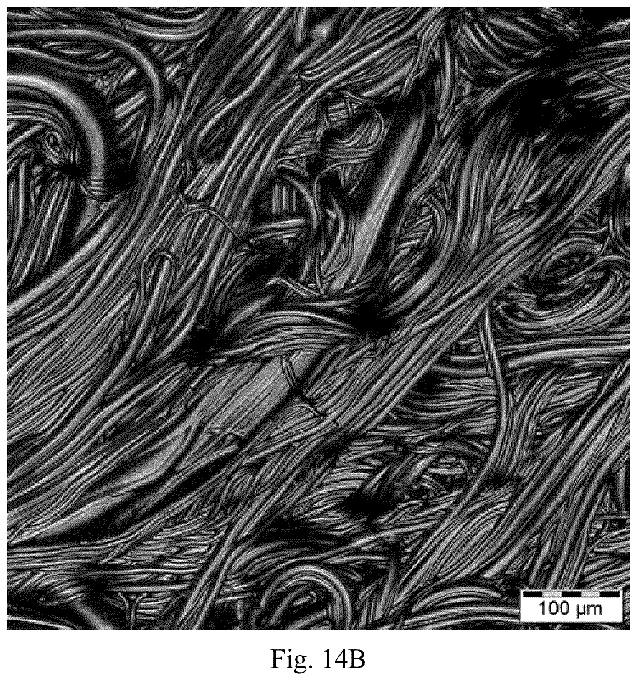

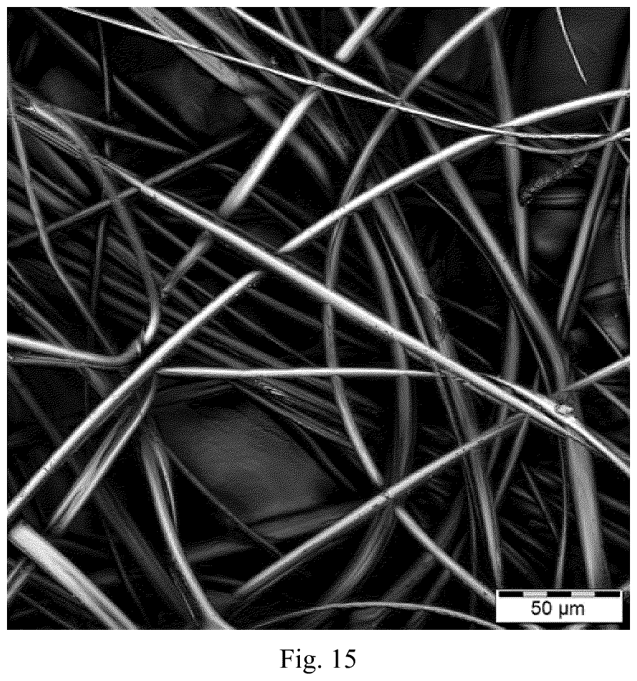

The procedure was the same as in Example 3, only with the differences, that the spinning solution 9 containing 1.87% by weight of sodium hyaluronan having the weight average molecular weight 2.12 MDa, and 1.87% by weight of hyaluronan 3-(2-furanyl) acryloyl ester having the weight average molecular weight 98 kDa and the substitution degree of 12.5%, was prepared from 2 grams of sodium hyaluronan having the weight average molecular weight 2.12 MDa, 2 grams of hyaluronan 3-(2-furanyl) acryloyl ester, 52.5 mL of 2-propanol 61.9 mL of water. The resulting fibers 12 having the diameter of 0.5-6 micrometers were removed from the collector 14 in the form of a bulky 3D structure (cotton wool) (FIG. 14A). The crosslinking of hyaluronan 3-(2-furanyl) acryloyl ester contained in the cotton wool was carried out in UVP Crosslinker CL-3000 (Analytik Jena), where the textile was exposed to radiation of a wavelength of 302 nm and energy intensity 350 μJ/cm² for 60 minutes. To verify the insolubility, the fabric sample was placed in demineralized water at temperature 22° C., after 2 weeks the fibrous structure was preserved (FIG. 14B).

### Example 16

The procedure was the same as in Example 3, only with the differences, that the spinning solution 9 containing 1.84% by weight of sodium hyaluronan having the weight average molecular weight 2.12 MDa and 3.27% by weight of hyaluronan chloramide having the weight average molecular weight 100 kDa and substitution degree of 96% was prepared from 1.8 grams of sodium hyaluronan having the weight average molecular weight 2.12 MDa, 3.2 grams of hyaluronan chloramide, 47.3 mL of 2-propanol, and 55.7 mL of water. The resulting fibers 12 having a diameter of 0.25-11 micrometers were obtained from the collector 14 in the form of a bulky 3D structure (cotton wool).

### Example 17

The procedure was the same as in Example 3, only with the differences, that the spinning solution 9 containing 1.67% by weight of sodium hyaluronan having the weight average molecular weight 2.05 MDa, 5.02% by weight of sodium hyaluronan having the weight average molecular weight 85 kDa, and 0.418% by weight of calcium chloride was prepared from 1.8 grams of sodium hyaluronan having the weight average molecular weight 2.05 MDa, 5.4 grams of sodium hyaluronan having the weight average molecular weight 85 kDa, 0.45 grams of calcium chloride, and 100 mL of water. The spinning solution 9 was extruded with the rate of 0.06 m¹/min. The resulting fibers 12 having a diameter of 0.3-3 micrometers were obtained from the collector 14 in the form of a bulky 3D structure (cotton wool).

### Example 18

The procedure was the same as in Example 3, only with the differences, that the spinning solution 9 containing 1.81% by weight of sodium hyaluronan having the weight average molecular weight 2.12 MDa, 3.62% by weight of sodium hyaluronan having the weight average molecular weight 30 kDa, 0.05% by weight of Ponceau 4R and 0.0025% by weight of Patent Blue VF was prepared from 4 grams of sodium hyaluronan having the weight average molecular weight 2.12 MDa, 8 grams of sodium hyaluronan having the weight average molecular weight 30 kDa, 111 milligrams of Ponceau 4R, 5.6 milligrams of Patent Blue VF, 93.1 mL of 2-propanol, and 135.8 mL of water. The spinning solution 9 was extruded with the rate of 0.14 mL/min. The resulting fibers 12 were carried by the air stream to the collector 14 in the shape of a cylinder with a diameter of 15 cm rotating at the speed of 2 revolutions per minute and located at a distance of 55 cm from the extrusion needle. The surface of the collector 14 was covered with a low-density polyethylene (LDPE) foil. The resulting non-woven textile which can be easily removed from the collector 14 has an area weight of 6 g/m² and is formed by fibers 12 having the diameter of 1-20 micrometers.

### Example 19

The procedure was the same as in Example 3, only with the differences, that the spinning solution 9 containing 1.42% by weight of sodium hyaluronan having the weight average molecular weight 2.05 MDa, 2.83% by weight of sodium hyaluronan having the weight average molecular weight 30 kDa, and 1.42% by weight of polyethylene oxide having the viscosity average molecular weight 1 MDa was prepared from 1.5 grams of sodium hyaluronan having the weight average molecular weight 2.05 MDa, 3 grams of sodium hyaluronan having the weight average molecular weight 30 kDa, 1.5 grams of polyethylene oxide having the viscosity average molecular weight 1 MDa, and 100 mL of water. The spinning solution 9 was extruded at a rate of 0.12 m¹/min. The resulting fibers 12 having a diameter of 1.6-4 micrometers were obtained from the collector 14 in the form of a bulky 3D structure (cotton wool).

### Example 20

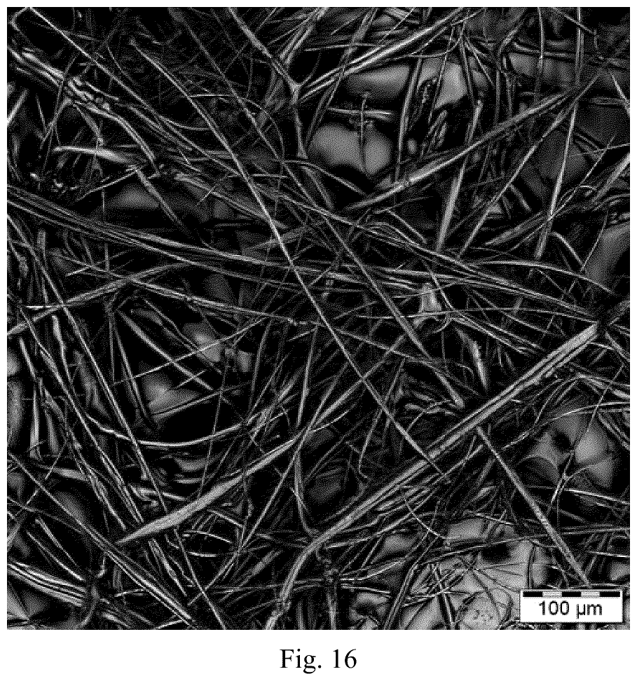

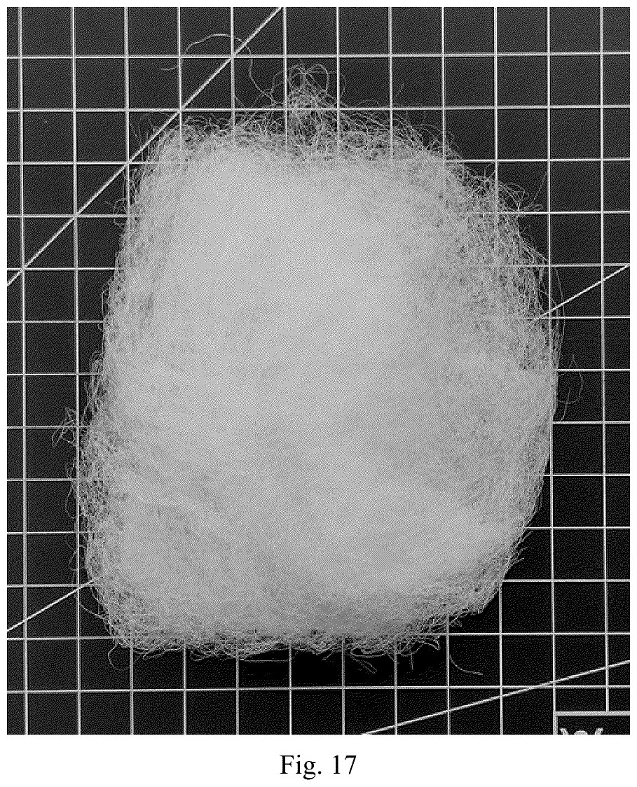

The procedure was the same as in Example 2, only with the differences, that the spinning solution 9 containing 1.83% by weight of sodium hyaluronan having the weight average molecular weight 2.12 MDa, 1.83% by weight of sodium hyaluronan having the weight average molecular weight 85 kDa, and 0.91% by weight of oxycellulose (Okcel Ca-L, Synthesia a.s., Czech Republic) was prepared using 2 grams of sodium hyaluronan having the weight average molecular weight 2.12 MDa, 2 grams of sodium hyaluronan having the weight average molecular weight 85 kDa, 1 gram of oxycellulose, 46.5 mL of 2-propanol, and 67.9 mL of water. The spinning solution 9 was extruded with the rate of 0.24 m¹/min. The air nozzle 5 blew the drying air with the volume flow of 400 L/min, which corresponds to the average air velocity of 32 m/s. The resulting fibers 12 have diameter of 1-12 micrometers and were removed from the collector 14 in the form of a bulky 3D structure (cotton wool) (FIG. 16).

### Example 21

The procedure was the same as in Example 1, only with the differences, that the spinning solution 9 containing 1.81% by weight of sodium hyaluronan having the weight average molecular weight 2.12 MDa, 3.62% by weight of sodium hyaluronan having the weight average molecular weight 30 kDa, 0.05% by weight of Ponceau 4R, and 0.0025% by weight of Patent Blue VF was prepared from 4 grams of sodium hyaluronan having the weight average molecular weight 2.12 MDa, 8 grams of sodium hyaluronan having the weight average molecular weight 30 kDa, 111 milligrams of Ponceau 4R, 5.6 milligrams of Patent Blue VF, 93.1 mL of 2-propanol, and 135.8 mL of water. The spinning solution 9 was extruded with the rate of 0.4 mL/min. The air nozzle 5 blew the drying air of the temperature of 180° C. and absolute humidity of 4 g/m³ with the volume flow of 280 L/min, which corresponds to an average air velocity of 17 m/s. The drying air is directed by a cylinder 13 located coaxially with the extrusion needle, wherein the cylinder 13 having the internal diameter of 3.7 cm and the length of 27.5 cm started at 1 cm below the end of the extrusion needle. The collector 14 was located 100 cm below the extrusion needle. The resulting fibers 12 having the diameter of 3-17 micrometers were removed from the collector 14 in the form of a non-woven textile having an area weight of 11 g/m².

### Example 22

The procedure was the same as in Example 3, only with the differences, that the spinning solution 9 containing 1.75% by weight of sodium hyaluronan having the weight average molecular weight 2.05 MDa and 7.00% by weight of sodium hyaluronan having the weight average molecular weight 30 kDa was prepared from 2 grams of sodium hyaluronan having the weight average molecular weight 2.05 MDa, 8 grams of sodium hyaluronan having the weight average molecular weight 30 kDa, 46.5 mL of 2-propanol, and 67.9 mL of water. The spinning solution 9 was extruded through the extrusion piece 11 which was an extrusion needle having an inner diameter of 410 micrometers with the rate of 0.2 m¹/min. The resulting fibers 12 having a diameter of 10-52 micrometers were obtained from the collector 14 in the form of a bulky 3D structure (cotton wool) (FIG. 17).

### Example 23

The procedure was the same as in Example 2, only with the differences, that the spinning solution 9 containing 1.81% by weight of sodium hyaluronan having the weight average molecular weight 2.12 MDa, 3.62% by weight of sodium hyaluronan having the weight average molecular weight 30 kDa, 0.05% by weight of Ponceau 4R, and 0.0025% by weight of Patent Blue VF was prepared from 4 grams of sodium hyaluronan having the weight average molecular weight 2.12 MDa, 8 grams of sodium hyaluronan having the weight average molecular weight 30 kDa, 111 milligrams of Ponceau 4R, 5.6 milligrams of Patent Blue VF, 93.1 mL of 2-propanol, and 135.8 mL of water. The extrusion piece 11 was an extrusion needle which has 4 capillaries 20 mm long with an internal diameter of 200 micrometers and the blunt ends of the capillaries formed the vertices of a square with a side of 4.5 mm. The spinning solution 9 was extruded at a rate of 0.2 mL/min per one capillary. The air nozzle 5 blew the drying air with the volume flow of 400 L/min, which corresponds to an average air velocity of 32 m/s. The resulting fibers 12 having the diameter of 2.5-25 micrometers were removed from the collector 14 in the form of a bulky 3D structure (cotton wool).

### Example 24

The procedure was the same as in Example 2, only with the differences, that the spinning solution 9 containing 1.67% by weight of sodium hyaluronan having the weight average molecular weight 2.12 MDa and 9.28% by weight of sodium hyaluronan having the weight average molecular weight 30 kDa was prepared from 2 grams of sodium hyaluronan having the weight average molecular weight 2.12 MDa, 11.1 grams of sodium hyaluronan having the weight average molecular weight 30 kDa, 34.0 mL of 2-propanol, and 80.0 mL of water. The spinning solution 9 was extruded with the rate of 0.7 m¹/min through the extrusion piece 11 which was a conical extrusion needle Tecdia ARQUE-S having an internal diameter of 100 micrometers. The air nozzle 5 blew the drying air with the volume flow of 400 L/min, which corresponds to an average air velocity of 32 m/s. The resulting fibers 12 having a diameter of 5-51 micrometers were obtained from the collector 14 in the form of a porous non-woven textile.

### Example 25

The procedure was the same as in Example 3, only with the differences, that the air having the temperature of 33° C. blew through the main outlet opening 4 in the center of the air nozzle 5 in which the extrusion piece 11 was coaxially arranged, the extrusion piece 11 being an extrusion needle. The volume flow of the air was 50 l/min, which corresponds to the average air velocity of 118 m/s. The heated air had a temperature of 231° C. and the volume flow thereof was 100 l/min, which corresponds to the average air velocity 14 m/s. The resulting fibers 12 having a diameter of 1-6.5 micrometers were obtained from the collector 14 in the form of a bulky 3D structure (cotton wool).

### Example 26

The procedure was the same as in Example 3, only with the difference, that the heated air had a temperature of 96° C. The resulting fibers 12 having a diameter of 0.9-5.2 micrometers were obtained from the collector 14 in the form of a bulky 3D structure (cotton wool).

### Example 27

The procedure was the same as in Example 3, only with the differences, that the heated air had a temperature of 96° C., and the volume flow thereof was 400 l/min, which corresponds to the average air velocity 56 m/s. The resulting fibers 12 having a diameter of 0.6-3.5 micrometers were obtained from the collector 14 in the form of a bulky 3D structure (cotton wool).

### Example 28

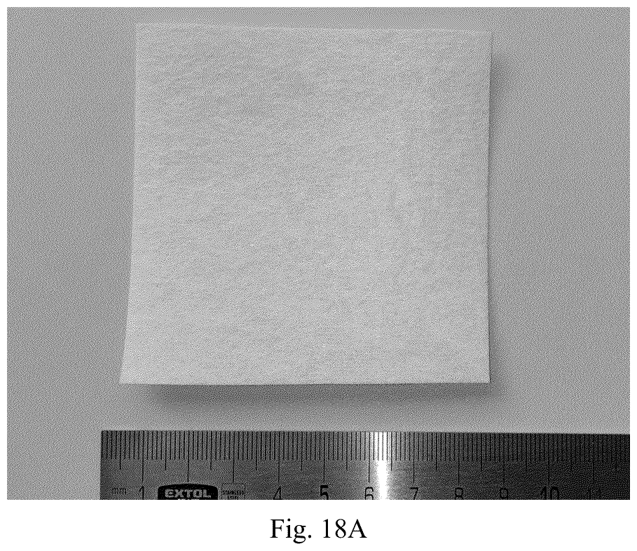

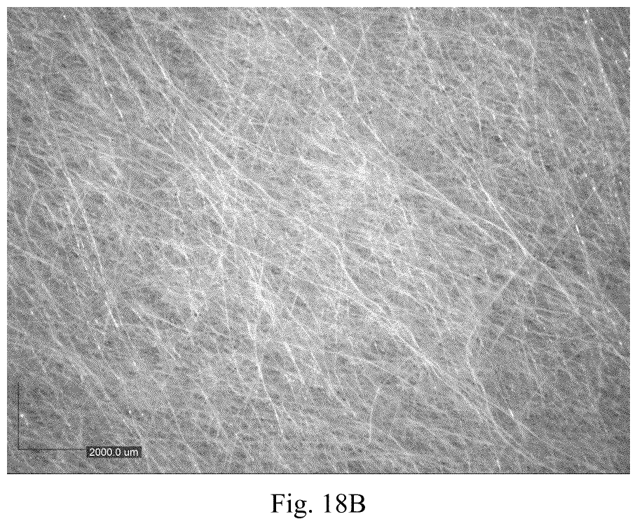

The procedure was the same as in Example 3, only with the differences, that the spinning solution 9 containing 1.75% by weight of sodium hyaluronan having the weight average molecular weight 2.05 MDa and 7.00% by weight of sodium hyaluronan having the weight average molecular weight 30 kDa was prepared from 2 grams of sodium hyaluronan having the weight average molecular weight 2.05 MDa, 8 grams of sodium hyaluronan having the weight average molecular weight 30 kDa, 46.5 mL of 2-propanol, and 67.9 mL of water. The spinning solution 9 was extruded through a blunt extrusion needle having the inner diameter of 210 micrometers, with the rate of 0.28 m¹/min. The resulting fibers 12 were carried by the air stream to the collector 14 in the shape of a cylinder with a diameter of 15 cm rotating at the speed of 6 revolutions per minute and located 74 cm under the extrusion needle. The surface of the collector 14 was covered with a polyester non-reinforced knitted fabric Zuzana (manufacturer: SILK & PROGRESS). The resulting non-woven fabric has the area weight of 98 g/m² and is formed by fibers 12 of diameter 6-36 micrometers (FIGS. 18A, 18B). The nonwoven fabric thus prepared is removed from the collector 14 together with the polyester fabric on which it is deposited, and can be used as such, as the resulting two-layer product, or the nonwoven fabric can be removed from the polyester fabric

### Example 29

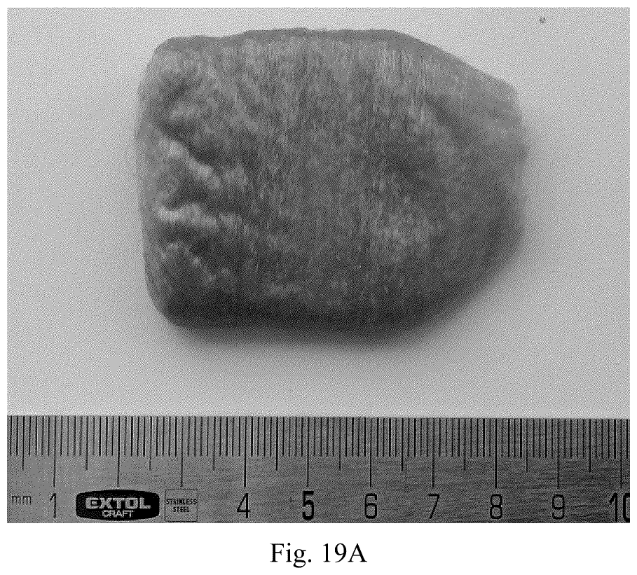

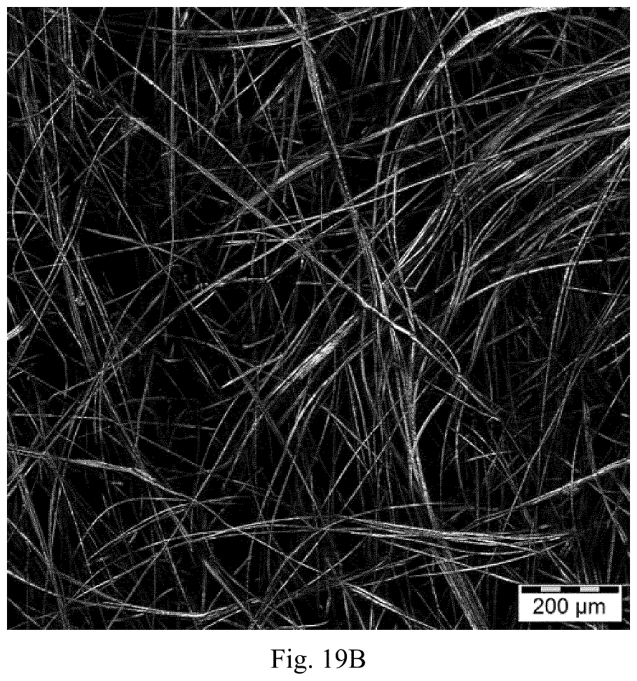

The procedure was the same as in Example 3, only with the differences, that the spinning solution 9 containing 1.81% by weight of sodium hyaluronan having the weight average molecular weight 2.12 MDa, 3.62% by weight of sodium hyaluronan having the weight average molecular weight 30 kDa, 0.05% by weight of Ponceau 4R, and 0.0025% by weight of Patent Blue VF was prepared from 4 grams of sodium hyaluronan having the weight average molecular weight 2.12 MDa, 8 grams of sodium hyaluronan having the weight average molecular weight 30 kDa, 111 milligrams of Ponceau 4R, 5.6 milligrams of Patent Blue VF, 93.1 mL of 2-propanol, and 135.8 mL of water. The spinning solution 9 was extruded with the rate of 0.12 mL/min. A Biotronik Actros DR pacemaker mounted on the axis of the collector 14 rotating at the speed of 13.5 revolutions per minute at a depth of 45 cm below the extrusion needle was used as the collector 14. The resulting non-woven fabric covering the pacemaker has the area weight of 31 g/m² and is formed by fibers 12 having a diameter of 1-17 micrometers (FIG. 19A; FIG. 19B).

### Example 30

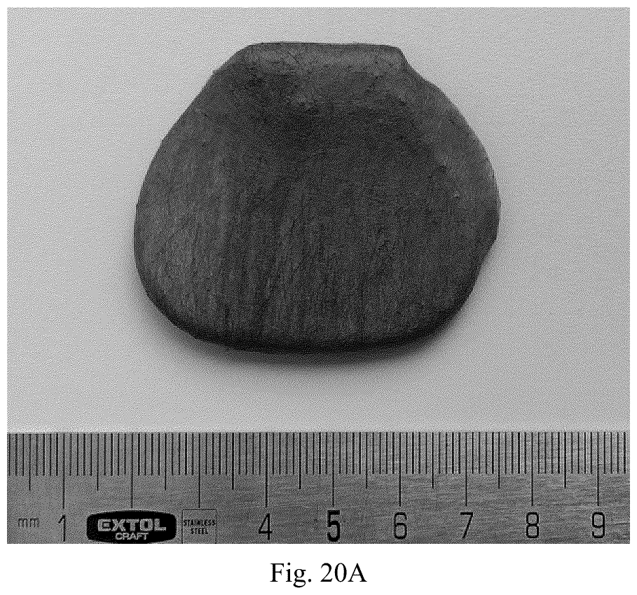

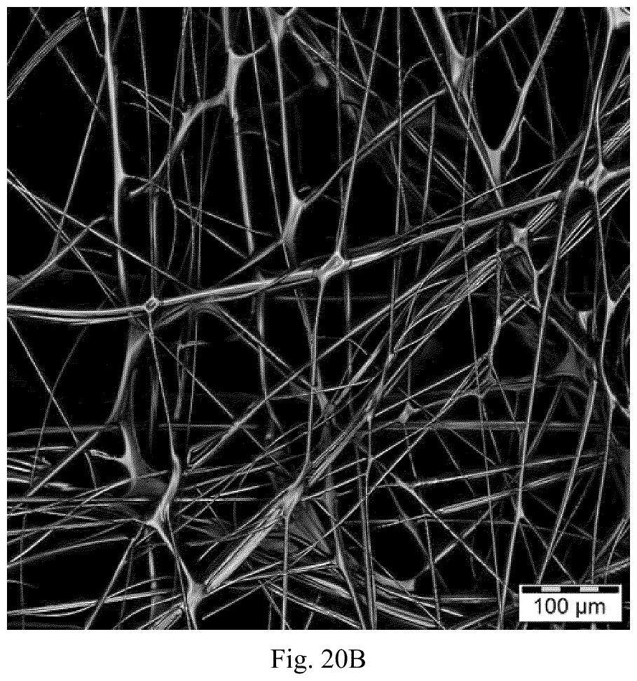

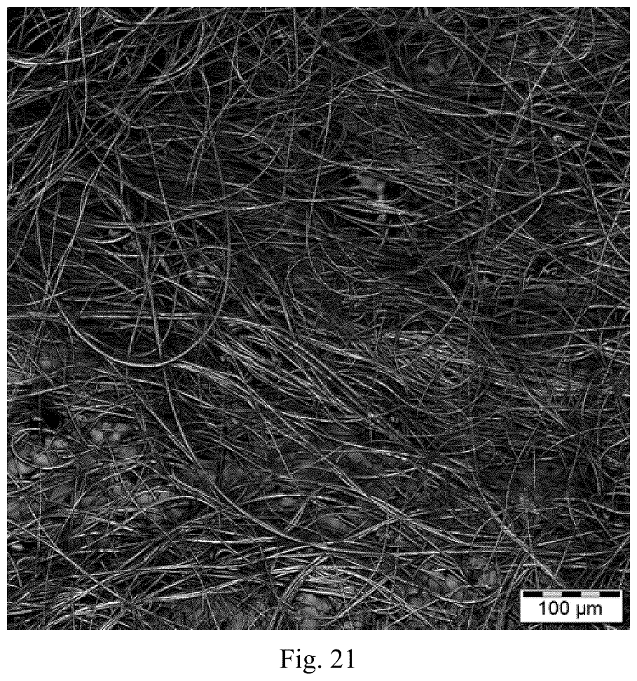

The procedure was the same as in Example 29, only with the difference, that the spinning solution 9 was extruded at the rate of 0.21 m¹/min. The resulting non-woven fabric covering the pacemaker has the area weight of 35 g/m² and is formed by fibers 12 having a diameter of 1-26 micrometers (FIGS. 20A, 20B).

### Example 31

The procedure was the same as in Example 3, only with the differences, that the spinning solution 9 was extruded with the rate of 0.08 mL/min. The air with a temperature of 29° C. was flowing through the main outlet opening 4 in the center of air nozzle 5, in which an extrusion needle, i.e., the extrusion piece 11, was located coaxially, with the volume flow of 50 L/min, corresponding to the average air velocity of 118 m/s. No air flowed from the two additional outlet openings 4a, 4b of the nozzle 5. The resulting fibers 12 having a diameter of 0.9-4.3 micrometers were obtained from the collector 14 in the form of a bulky 3D structure (cotton wool) (FIG. 21).

### Example 32

The procedure was the same as in Example 4, only with the differences that the spinning solution 9 was extruded with the rate of 0.1 mL/min, the volume flow of the drying air was 150 l/min, which corresponds to the average air velocity of 199 m/s, and the distance between the collector 14 and the extrusion needle was 15 cm. The resulting fibers 12 having a diameter of 0.25-3 micrometers were obtained from the collector 14 in the form of a thin layers which were packed into a 3D structure (cotton wool).

1. A method of production of fibers based on a polymer selected from hyaluronic acid, a water-soluble metal compound thereof, a derivative thereof, or combinations thereof by dry spinning and/or solution blow spinning, the method comprising the steps of:
providing a spinning solution comprising:
from about 0.08 to about 3% by weight of hyaluronic acid, a water-soluble metal or non-metal salt thereof or a water-soluble mixture of metal and/or non-metal salts of hyaluronic acid and/or hyaluronic acid derivative, having a high molecular weight in the range of from about 1.1 MDa to about 2.5 MDa,
from about 0.1 to about 22% by weight of hyaluronic acid and/or a water-soluble metal or non-metal salt thereof or a water-soluble mixture of metal and/or non-metal salts of hyaluronic acid and/or hyaluronic acid derivative, having a low molecular weight in the range of from about 5 kDa to about 750 kDa,
wherein the water-soluble metal or non-metal salt of hyaluronic acid is selected from the group comprising Na⁺, K⁺, Lit, Ca²⁺, Mg²⁺, Ag⁺, Au⁺, Zn²⁺, Cu²⁺, and/or NH⁴⁺ salt of hyaluronic acid, and the hyaluronic acid derivative is selected from the group comprising hyaluronan chloramide, hyaluronan 3-(2-furanyl) acryloyl ester, hyaluronan tyramine, hyaluronan benzyl ester, hyaluronan ethyl ester, and acylated hyaluronan derivatives selected from the group comprising capronoyl, capryloyl, caprinoyl, myristoyl, palmitoyl, stearoyl, and/or oleoyl hyaluronan,
from about 0 to about 50% by weight of organic solvent, and
from about 48 to about 95% by weight of water, and
extruding the spinning solution by an extrusion piece having at least one opening having a diameter of from about 80 to about 410 μm with a rate of from about 0.01 to about 0.7 mL/min into a drying air stream to form fibers that are carried to a collector.

2. The method of production of fibers of claim 1, wherein the weight average molecular weight of hyaluronic acid and/or a water-soluble metal or non-metal salt thereof and/or hyaluronic acid derivative, having a high molecular weight is in the range from about 1.8 MDa to about 2.5 MDa, and the weight average molecular weight of hyaluronic acid and/or a water-soluble metal or non-metal salt thereof and/or hyaluronic acid derivative having a low molecular weight is in the range from about 5 kDa to about 100 kDa.

3. The method of production of fibers of claim 1, wherein the spinning solution comprises:
from about 1 to about 2% by weight of hyaluronic acid and/or a metal or non-metal salt thereof or a mixture of metal and/or non-metal salts of hyaluronic acid and/or hyaluronic acid derivative having a high molecular weight,
from about 1 to about 8% by weight of hyaluronic acid and/or a metal or non-metal salt thereof or a mixture of metal and/or non-metal salts of hyaluronic acid and/or hyaluronic acid derivative having a low molecular weight,
from about 25 to about 40% by weight of 2-propanol, and
from about 58 to about 73% by weight of water.

4. The method of production of fibers of claim 1, wherein the spinning solution comprises:
from about 0.08 to about 3% by weight of hyaluronic acid and/or a water-soluble metal or non-metal salt thereof or a water-soluble mixture of metal and/or non-metal salts of hyaluronic acid and/or hyaluronic acid derivative having a high molecular weight in the range from about 1.1 MDa to about 2.5 MDa,
from about 0.1 to about 22% by weight of hyaluronic acid derivative with a low molecular weight in the range from about 5 kDa to about 750 kDa, wherein the derivative is hyaluronan chloramide, or a mixture of hyaluronic acid derivative, which is hyaluronan chloramide, and native hyaluronic acid, wherein the substitution degree of hyaluronan chloramide is in the range from about 0.1 to about 100%,
from about 0 to about 50% by weight of organic solvent, and
from about 48 to about 95% by weight of water.

5. The method of production of fibers of claim 1, wherein the spinning solution comprises:
from about 0.08 to about 3% by weight of hyaluronic acid and/or a water-soluble metal or non-metal salt thereof or a water-soluble mixture of metal and/or non-metal salts of hyaluronic acid and/or hyaluronic acid derivative having a high molecular weight in the range from about 1.1 MDa to about 2.5 MDa,
from about 0.1 to about 22% by weight of hyaluronic acid derivative having a low molecular weight in the range from about 5 kDa to about 750 kDa, wherein the derivative is hyaluronan 3-(2-furanyl) acryloyl ester, or a mixture of hyaluronic acid derivative, which is hyaluronan 3-(2-furanyl) acryloyl ester, and native hyaluronic acid, wherein the substitution degree of hyaluronan by 3-(2-furanyl) acryloyl is in the range from about 0.1 to about 20%, and wherein the proportion of hyaluronan 3-(2-furanyl) acryloyl ester in the mixture with native HA is at least about 0.1%,
from about 0 to 5 about 0% by weight of organic solvent, and
from about 48 to about 95% by weight of water, wherein the method further comprises the step of subsequently crosslinking the fibers by radiation with a wavelength in the range from about 280-750 nm for about 2-60 minutes.

6. The method of production of fibers of claim 1, wherein the organic solvent is selected from the group comprising methanol, tetrahydrofuran, methyl acetate, methyl ethyl ketone, 1,2-dimethoxyethane, acetonitrile, isopropyl alcohol, 1-propanol, ethanol and acetone.

7. The method of production of fibers of claim 1, further comprising the step of preparing the spinning solution, wherein preparing the spinning solution comprises:
dispersing the hyaluronic acid, the water-soluble metal or non-metal salt thereof, the hyaluronic acid derivative or salt thereof, or the combination thereof in an organic solvent to form a dispersion,
adding water to the resulting dispersion with thorough mixing, and
stirring the dispersion for about 1 to about 24 hours at a temperature of about 20 to about 30° C. until the polymer is completely dissolved, to form the solution.

8. The method of production of fibers of claim 1, further comprising the steps of:
filling a cartridge with the prepared spinning solution,
sealing the cartridge; and
connecting the cartridge to a source of compressed air having a pressure of about +500 to about +700 kPa for about 1 to about 8 hours.

9. The method of production of fibers claim 1, wherein the spinning solution further comprises another polymer selected from the group comprising polyethylene oxide, polyvinyl pyrrolidone, pullulan, carboxymethyl cellulose or oxycellulose, and/or one or more pharmaceutically and/or cosmetically acceptable low molecular weight substances selected from the group comprising antibacterial agents, antivirals, antifungals, drugs, vitamins, plant extracts, surfactants, peptides, and dyes.

10. The method of production of fibers of claim 1, wherein the drying air temperature is in the range from about 15 to about 250° C., and the absolute humidity of the drying air is in the range from about 0 to about 14 g/m³, and the drying air velocity is in the range from about 1.6 to about 315 m/s.

11. The method of production of fibers of claim 1, wherein the fibers are deposited on the collector covered with an inert material having a low surface energy, from which the fibers are easily removed, or with a textile, which remains a part of the final product.

12. The method of production of fibers of claim 1, wherein the fibers are deposited on the collector, which is an implantable medical device.

13. The method of production of fibers of claim 1, wherein the fibers have a diameter of about 100 nm to about 100 μm and form a non-woven 2D or 3D fabric on the collector, the fabric having an area weight of about 0.1 to about 120 g/m².

14. A device for carrying out the method of claim 1, comprising:
an extrusion piece (11) comprising a pass-through channel, which has an inlet opening for feeding a spinning solution (9) and at least one dispensing opening (11a) for dispensing the spinning solution (9),
an air nozzle (5), wherein the main outlet opening (4) for exiting air is arranged to direct the exiting air into the area surrounding the dispensing opening (11a) of the extrusion piece (11) parallel to the axis of the dispensing opening (11a) of the extrusion piece (11), the air nozzle (5) includes additional outlet openings (4a, 4b) that are arranged to direct the additional air flow to the area under the outlet opening (11a) into the air flow from the main outlet opening (4) at an angle of 30 to 60°, and
at least one cooling channel (15) for directing the cooling liquid through the air nozzle (5) walls.

15. The method of claim 4, wherein the substitution degree of hyaluronan chloramide is in the range from about 50 to about 100%.

16. The method of claim 9, wherein the peptides are selected from the group comprising antimicrobial peptides, wound healing promoting peptides, and hormonal peptides.

17. The method of claim 10, wherein the drying air temperature is in the range from about 15 to about 160° C., the absolute humidity of the drying air is in the range from about 0 to about 2 g/m³, and the drying air velocity is in the range from about 5 to about 200 m/s.

18. The method of claim 11, wherein the inert material having a low surface energy is selected from polytetrafluoroethylene, polyethylene, or combinations thereof, and wherein the textile is a polyester knitted fabric.

19. The method of claim 14, wherein the additional outlet openings (4a, 4b) are arranged to direct the additional air flow to the area under the outlet opening (11a) into the air flow from the main outlet opening (4) at an angle of from about 40 to about 50°.
